(19) United States
(12) Patent Application Publication
(10) Pub. No.: US 2024/0199645 A1
(43) Pub. Date: Jun. 20, 2024

(54) POLYMORPHIC FORMS OF (S)-12-FLUORO-4-(1-METHYL-1H-PYRAZOL-5-YL)-7A,8,13,14-TETRAHYDRO-7H-[1,2,4]TRIAZOLO[4',3':1,6]PYRIDO[3,2-B]BENZOFURO[4,3-FG][1,4]OXAZONINE

(71) Applicant: Fulcrum Therapeutics, Inc., Cambridge, MA (US)

(72) Inventors: Steven M. Mennen, Boston, MA (US); Colin Kelly, Halifax (CA)

(21) Appl. No.: 18/553,352

(22) PCT Filed: Mar. 31, 2022
(86) PCT No.: PCT/US2022/022895

§ 371 Date: Sep. 29, 2023

Related U.S. Application Data
(60) Provisional application No. 63/170,226, filed on Apr. 2, 2021.

(51) Int. Cl. C07D 498/22; A61K 31/437

(52) U.S. Cl. CPC C07D 498/22; A61K 31/437

(57) ABSTRACT

The disclosure is in part directed to crystalline forms of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

### CROSS-REFERENCE

This application claims priority to U.S. Provisional Application No. 63/170,226 filed Apr. 2, 2021, which is incorporated herein by reference in its entirety.

### BACKGROUND

Polycomb Repressive Complex 2 (PRC2) is a multi-subunit chromatin regulatory complex that functions in repression of gene expression and is dysregulated in many human diseases. PRC2 includes SUZ12 (suppressor of zeste 12), EED (embryonic ectoderm development) and the catalytic subunit, EZH2 (enhancer of zeste homolog 2), and represses genes by methylating histone H3 on lysine 27 (H3K27me3) at and around the promoter regions of genes. EED mediates repression of gene activity by binding to the H3K27me3 mark where it allosterically activates the methyltransferase activity of PRC2 (e.g., trimethylation of lysine 27 on histone H3 (H3K27me3). This critical component of chromatin regulation is involved in modulation of gene transcription and plays a key role in development, differentiation, and regeneration.

EED regulates PRC2 in the silencing of expression of genes and gene clusters involved in development, e.g., fetal orthologues (e.g., gamma globin), Hox genes, and in X chromosome inactivation. Aberrant expression of PRC2 has also been observed in various human cancers, for example, hepatocellular carcinoma, breast cancer, and prostate cancer. Thus, EED and/or PRC2 provides a pharmacological target for the treatment of diseases or disorders, for example, cancers and blood disorders, to impact transcription of specific target genes in, for example, blood and other tissues.

Polymorphism is the ability of a substance to crystallize in more than one crystal lattice arrangement. Crystallization, or polymorphism, can influence many aspects of the solid-state properties of a drug substance. A crystalline substance may differ considerably from an amorphous form, and different crystal modifications of a substance may differ considerably from one another in many respects including solubility, dissolution rate and/or bioavailability. Generally, it is difficult to predict whether a given compound will form any crystalline solid-state forms. It is even more difficult to predict the physical properties of these crystalline solid-state forms. Further, it can be advantageous to have a crystalline form of a therapeutic agent for certain formulations and/or for manufacturing processes.

### SUMMARY

The present disclosure is directed in part to crystalline forms of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

For example, disclosed herein is a crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine characterized by a powder X-ray diffraction pattern having a characteristic peak in degrees 2θ at about 9.8, for example, characterized by a powder X-ray diffraction pattern having characteristic peaks in degrees 2θ at about 9.6, 9.8, and 13.3, for example, characterized by a powder X-ray diffraction pattern having characteristic peaks in degrees 2θ at about 6.4, 8.3, 9.6, 9.8, 12.8, and 13.3, for example, characterized by a powder X-ray diffraction pattern having characteristic peaks in degrees 2θ at about 6.4, 8.3, 9.6, 9.8, 10.6, 12.8, 13.3, 17.2, 20.0, 21.3, 22.2, and 26.0.

(S)-12-fluoro-4-(1-methyl-1H-pyrazol-5-yl)-7a,8,13,14-tetrahydro-7H-[1,2,4]triazolo[4′,3′: 1,6]pyrido[3,2-b]benzofuro[4,3-fg][1,4]oxazonine is, for example, a modulator of EED and/or a modulator of PRC2, and is represented by:

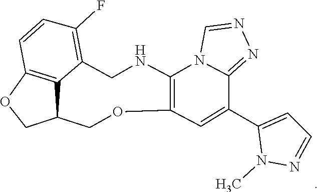 [SMILES: Cn1nccc1-c1cc2c(n3cnnc13)NCc1c(F)ccc3c1[C@H](CO3)CO2]

Further contemplated herein is a pharmaceutical composition comprising a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine and a pharmaceutically acceptable excipient, for example, a composition that is formulated for oral, subcutaneous or intravenous administration. Further contemplated herein is a drug substance comprising at least a detectable amount of a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine. For example, disclosed herein is a drug substance comprising substantially pure crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

Also provided herein is a method of treating a blood disorder (e.g., sickle cell disease or β-thalassemia) in a patient in need thereof, comprising administering to the patient an effective amount of a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine. For example, provided herein is a method of treating a blood disorder (e.g., sickle cell disease or β-thalassemia) in a patient in need thereof, comprising administering to the patient an effective amount of a pharmaceutical composition comprising a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

Further provided herein is a method of treating a cancer in a patient in need thereof, comprising administering to the patient an effective amount of a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine. For example, provided herein is a method of treating a cancer in a patient in need thereof, comprising administering to the patient an effective amount of a pharmaceutical composition comprising a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

### BRIEF DESCRIPTION OF THE DRAWINGS

FIG. 1 depicts the X-ray powder diffraction (XRPD) pattern of Form A of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 2 depicts the characterization of Form A by differential scanning calorimetry (DSC).

FIG. 3 depicts the X-ray powder diffraction (XRPD) pattern of Form B of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 4 depicts the characterization of Form B by differential scanning calorimetry (DSC).

FIG. 5 depicts the X-ray powder diffraction (XRPD) pattern of Form C of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 6 depicts the characterization of Form C by differential scanning calorimetry (DSC).

FIG. 7 depicts the X-ray powder diffraction (XRPD) pattern of Form D of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 8 depicts the X-ray powder diffraction (XRPD) pattern of Form E of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 9 depicts the characterization of Form E by differential scanning calorimetry (DSC).

FIG. 10 depicts the X-ray powder diffraction (XRPD) pattern of Form F of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 11 depicts the X-ray powder diffraction (XRPD) pattern of Form G of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 12 depicts the characterization of Form G by differential scanning calorimetry (DSC).

FIG. 13 depicts the X-ray powder diffraction (XRPD) pattern of Form H of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 14 depicts the X-ray powder diffraction (XRPD) pattern of Form I of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 15 depicts the X-ray powder diffraction (XRPD) pattern of Form J of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 16 depicts the X-ray powder diffraction (XRPD) pattern of Form K of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 17 depicts the X-ray powder diffraction (XRPD) pattern of Form L of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 18 depicts the X-ray powder diffraction (XRPD) pattern of Form M of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 19 depicts the X-ray powder diffraction (XRPD) pattern of Form N of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 20 depicts the characterization of Form N by differential scanning calorimetry (DSC).

FIG. 21 depicts the X-ray powder diffraction (XRPD) pattern of Form O of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 22 depicts the X-ray powder diffraction (XRPD) pattern of Form P of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 23 depicts the characterization of Form P by differential scanning calorimetry (DSC).

FIG. 24 depicts the X-ray powder diffraction (XRPD) pattern of Form Q of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 25 depicts the characterization of Form Q by differential scanning calorimetry (DSC).

FIG. 26 depicts the X-ray powder diffraction (XRPD) pattern of Form R of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 27 depicts the X-ray powder diffraction (XRPD) pattern of Form S of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 28 depicts the X-ray powder diffraction (XRPD) pattern of Form T of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 29 depicts the X-ray powder diffraction (XRPD) pattern of Form U of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 30 depicts the characterization of Form U by differential scanning calorimetry (DSC).

FIG. 31 depicts the X-ray powder diffraction (XRPD) pattern of Form V of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 32 depicts the characterization of Form V by differential scanning calorimetry (DSC).

FIG. 33 depicts the X-ray powder diffraction (XRPD) pattern of Form W of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 34 depicts the X-ray powder diffraction (XRPD) pattern of Form X of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 35 depicts the X-ray powder diffraction (XRPD) pattern of Form Y of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 36 depicts the X-ray powder diffraction (XRPD) pattern of Form Z of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 37 depicts the X-ray powder diffraction (XRPD) pattern of Form α of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 38 depicts the X-ray powder diffraction (XRPD) pattern of Form β of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 39 depicts the X-ray powder diffraction (XRPD) pattern of Form γ of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

FIG. 40 depicts the X-ray powder diffraction (XRPD) pattern of amorphous (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “crystalline form” refers to a crystal form or modification that can be characterized by analytical methods such as, e.g., X-ray powder diffraction (XRPD) and/or Differential scanning calorimetry (DSC). The crystalline compounds disclosed herein can exist in solvated as well as unsolvated forms with solvents such as water, ethanol, and the like. Unless otherwise indicated or inferred, it is intended that disclosed crystalline compounds include both solvated and unsolvated forms.

“Treating” includes any effect, e.g., lessening, reducing, modulating, or eliminating, that results in the improvement of the condition, disease, disorder and the like.

The term “disorder” refers to and is used interchangeably with, the terms “disease,” “condition,” or “illness,” unless otherwise indicated.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable excipient” or “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the present disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the present disclosure is desirably a mammal in which treatment, for example, of a cancer or a blood disorder is desired. “Modulation” includes antagonism (e.g., inhibition), agonism, partial antagonism and/or partial agonism.

In the present specification, the terms “effective amount” or “therapeutically effective amount” means the amount of the subject compound that will elicit the biological or medical response of a tissue, system or animal, (e.g. mammal or human) that is being sought by the researcher, veterinarian, medical doctor or other clinician. The compounds of the present disclosure are administered in therapeutically effective amounts to treat a disease. Alternatively, a therapeutically effective amount of a compound is the quantity required to achieve a desired therapeutic and/or prophylactic effect.

The term “pharmaceutically acceptable salt(s)” as used herein refers to salts of basic groups that may be present in compounds used in the compositions. Compounds included in the present compositions that are basic in nature are capable of forming a wide variety of salts with various inorganic and organic acids.

The term “and/or” is used in this disclosure to mean either “and” or “or” unless indicated otherwise.

As used herein, the words “a” and “an” are meant to include one or more unless otherwise specified. For example, the term “an agent” encompasses both a single agent and a combination of two or more agents.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. The term “about” in the context of peaks at degrees 2θ means that there is an uncertainty in the measurements of the 2θ of ±0.5 (expressed in 2θ) or that there is an uncertainty in the measurements of the 2θ of ±0.2 (expressed in 2θ).

### Crystalline Forms

The present disclosure is directed in part to crystalline forms of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

For example, disclosed herein is a crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine characterized by a powder X-ray diffraction pattern having a characteristic peak in degrees 2θ at about 9.8 (referred to herein as “Form E”).

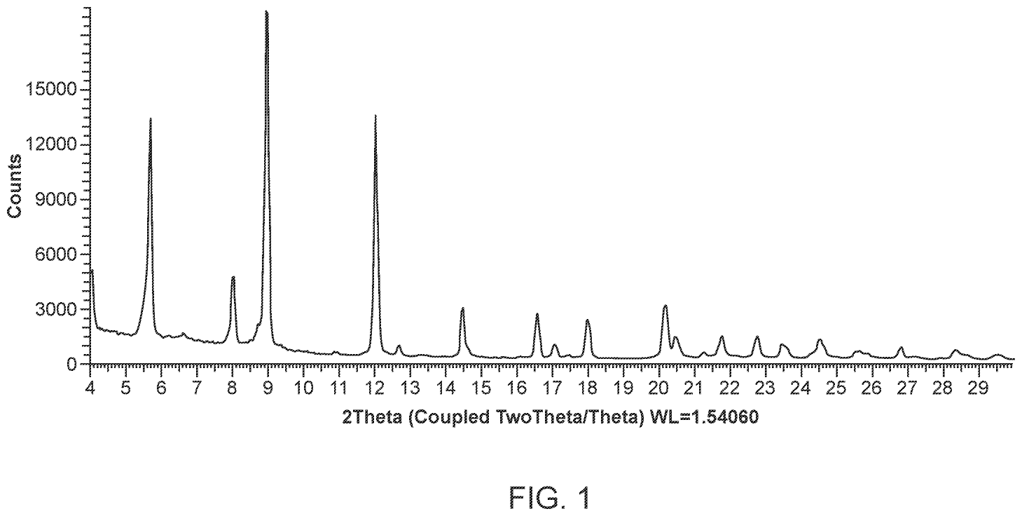

In one embodiment, the crystalline Form E of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 6.4, is characterized by a powder X-ray diffraction pattern that has a characteristic peak in degrees 2θ at about 8.3, is characterized by a powder X-ray diffraction pattern that has a characteristic peak in degrees 2θ at about 9.6, is characterized by a powder X-ray diffraction pattern that has a characteristic peak in degrees 2θ at about 10.6, is characterized by a powder X-ray diffraction pattern that has a characteristic peak in degrees 2θ at about 12.8, is characterized by a powder X-ray diffraction pattern that has a characteristic peak in degrees 2θ at about 13.3, is characterized by a powder X-ray diffraction pattern that has a characteristic peak in degrees 2θ at about 17.2, is characterized by a powder X-ray diffraction pattern that has a characteristic peak in degrees 2θ at about 20.0, is characterized by a powder X-ray diffraction pattern that has a characteristic peak in degrees 2θ at about 21.3, is characterized by a powder X-ray diffraction pattern that has a characteristic peak in degrees 2θ at about 22.2, and/or is characterized by a powder X-ray diffraction pattern that has a characteristic peak in degrees 2θ at about 26.0. In another embodiment, crystalline Form E is characterized by a powder X-ray diffraction pattern having at least one or more characteristic peaks in degrees 2θ at about 9.6, 9.8, and 13.3. In a further embodiment, crystalline Form E is characterized by a powder X-ray diffraction pattern having at least one or more characteristic peaks in degrees 2θ at about 6.4, 8.3, 9.6, 9.8, 12.8, and 13.3. In yet another embodiment, crystalline Form E is characterized by a powder X-ray diffraction pattern having at least one or more characteristic peaks in degrees 2θ at about 6.4, 8.3, 9.6, 9.8, 10.6, 12.8, 13.3, 17.2, 20.0, 21.3, 22.2, and 26.0. For example, a contemplated crystalline form has a powder X-ray diffraction pattern shown in FIG. 1. In one embodiment, the powder X-ray diffraction pattern of the crystalline form was obtained using Cu Kα radiation.

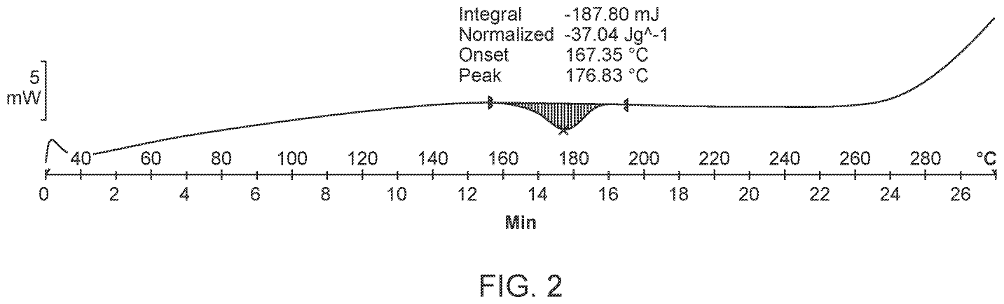

The contemplated crystalline Form E of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a differential scanning calorimetry (DSC) profile showing a characteristic endotherm with an onset of about 272° C. and a peak of about 274° C. Form E, for example, may be characterized by the differential scanning calorimetry profile shown in FIG. 2.

The contemplated crystalline Form E of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a thermogravimetric analysis (TGA) profile showing a mass loss of about 0.86 wt. % up to melting. In some embodiments, crystalline Form E may be characterized by a dynamic vapor sorption (DVS) profile showing a mass change of about 0.23 wt. % between about 2 to about 92% relative humidity (RH) at 25° C. In other embodiments, crystalline Form E may be characterized by optical microscopy showing a needle-like morphology.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 9.0 (referred to herein as “Form A”), is disclosed herein.

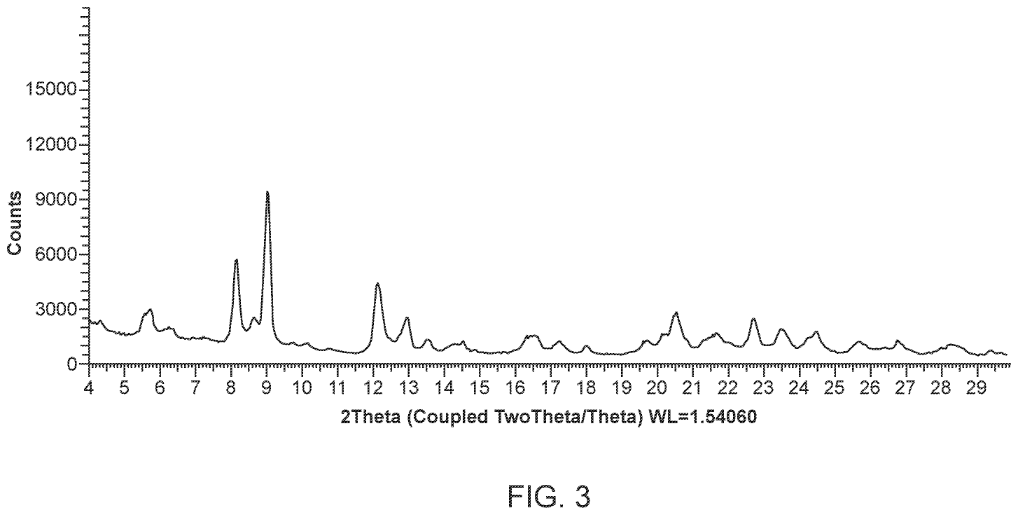

In one embodiment, the crystalline Form A of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 5.7, is characterized by a powder X-ray diffraction pattern that has a characteristic peak in degrees 2θ at about 8.1, is characterized by a powder X-ray diffraction pattern that has a characteristic peak in degrees 2θ at about 8.6, is characterized by a powder X-ray diffraction pattern that has a characteristic peak in degrees 2θ at about 12.0, is characterized by a powder X-ray diffraction pattern that has a characteristic peak in degrees 2θ at about 14.4, is characterized by a powder X-ray diffraction pattern that has a characteristic peak in degrees 2θ at about 16.5, is characterized by a powder X-ray diffraction pattern that has a characteristic peak in degrees 2θ at about 17.9, is characterized by a powder X-ray diffraction pattern that has a characteristic peak in degrees 2θ at about 20.1, is characterized by a powder X-ray diffraction pattern that has a characteristic peak in degrees 2θ at about 20.4, is characterized by a powder X-ray diffraction pattern that has a characteristic peak in degrees 2θ at about 21.6, and/or is characterized by a powder X-ray diffraction pattern that has a characteristic peak in degrees 2θ at about 24.4. In yet another embodiment, crystalline Form A is characterized by a powder X-ray diffraction pattern having at least one or more characteristic peaks in degrees 2θ at about 5.7, 8.1, 8.6, 9.0, 12.0, 14.4, 16.5, 17.9, 20.1, 20.4, 21.6, and 24.4. For example, a contemplated crystalline form has a powder X-ray diffraction pattern shown in FIG. 3. In one embodiment, the powder X-ray diffraction pattern of the crystalline form was obtained using Cu Kα radiation.

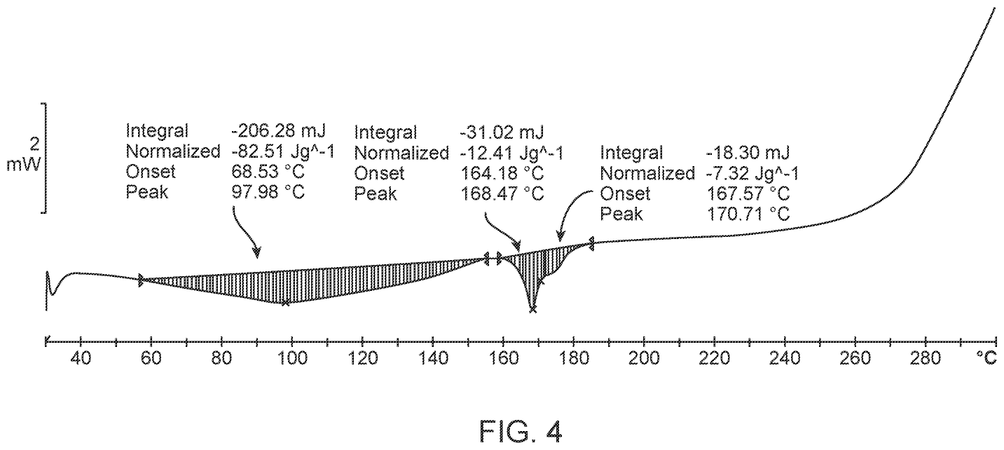

The contemplated crystalline Form A of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a differential scanning calorimetry (DSC) profile showing a characteristic endotherm with an onset of about 167° C. and a peak of about 177° C. Form A, for example, may be characterized by the differential scanning calorimetry profile shown in FIG. 4.

The contemplated crystalline Form A of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a thermogravimetric analysis (TGA) profile showing a mass loss of 3.64 wt. % up to 160° C. and an additional loss of 1.97 wt. % from 160 to 250° C. (5.61 wt. % total). In some embodiments, crystalline Form A may be characterized by a dynamic vapor sorption (DVS) profile showing a mass change of about 6.34 wt. % (about 1.48 eq. water) between about 2 to about 92% relative humidity (RH) at 25° C. In other embodiments, crystalline Form A may be characterized by optical microscopy showing a mixture of irregular and needle-like morphologies.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 9.0 (referred to herein as “Form B”), is disclosed herein.

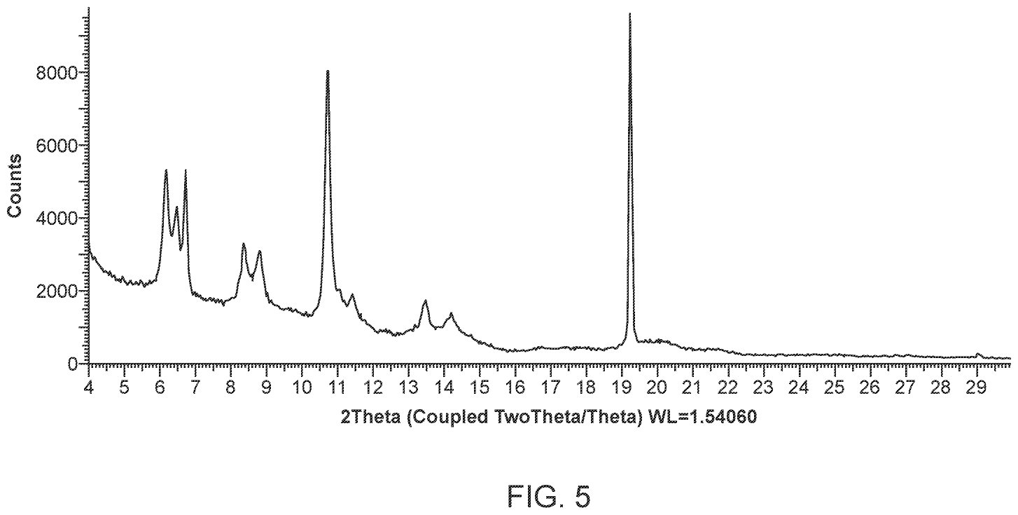

In one embodiment, the crystalline Form B of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 5.7, is characterized by a powder X-ray diffraction pattern that has a characteristic peak in degrees 2θ at about 8.1, is characterized by a powder X-ray diffraction pattern that has a characteristic peak in degrees 2θ at about 8.6, is characterized by a powder X-ray diffraction pattern that has a characteristic peak in degrees 2θ at about 12.1, is characterized by a powder X-ray diffraction pattern that has a characteristic peak in degrees 2θ at about 12.9, is characterized by a powder X-ray diffraction pattern that has a characteristic peak in degrees 2θ at about 16.5, is characterized by a powder X-ray diffraction pattern that has a characteristic peak in degrees 2θ at about 20.5, is characterized by a powder X-ray diffraction pattern that has a characteristic peak in degrees 2θ at about 21.7, is characterized by a powder X-ray diffraction pattern that has a characteristic peak in degrees 2θ at about 22.7, is characterized by a powder X-ray diffraction pattern that has a characteristic peak in degrees 2θ at about 23.5, and/or is characterized by a powder X-ray diffraction pattern that has a characteristic peak in degrees 2θ at about 24.5. In yet another embodiment, crystalline Form B is characterized by a powder X-ray diffraction pattern having at least one or more characteristic peaks in degrees 2θ at about 5.7, 8.1, 8.6, 9.0, 12.1, 12.9, 16.5, 20.5, 21.7, 22.7, 23.5, and 24.5. For example, a contemplated crystalline form has a powder X-ray diffraction pattern shown in FIG. 5. In one embodiment, the powder X-ray diffraction pattern of the crystalline form was obtained using Cu Kα radiation.

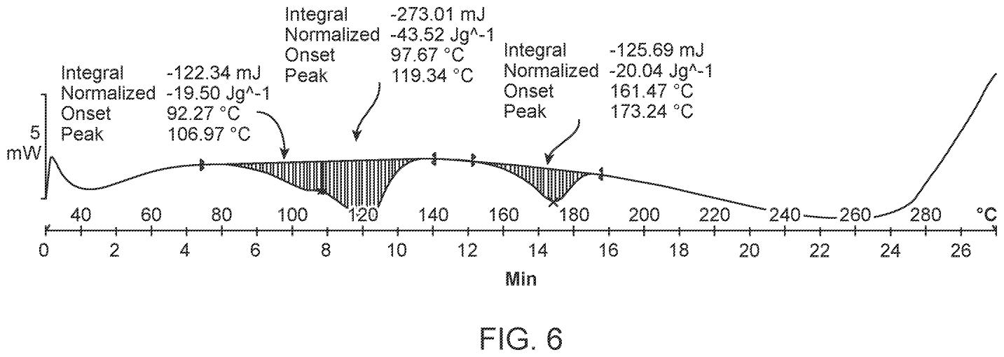

The contemplated crystalline Form B of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a differential scanning calorimetry (DSC) profile showing a characteristic endotherm with an onset of about 68° C. and a peak of about 98° C., a characteristic endotherm with an onset of about 164° C. and a peak of about 168° C., and a characteristic endotherm with an onset of about 168° C. and a peak of about 171° C. Form B, for example, may be characterized by the differential scanning calorimetry profile shown in FIG. 6.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 10.7 (referred to herein as “Form C”), is disclosed herein.

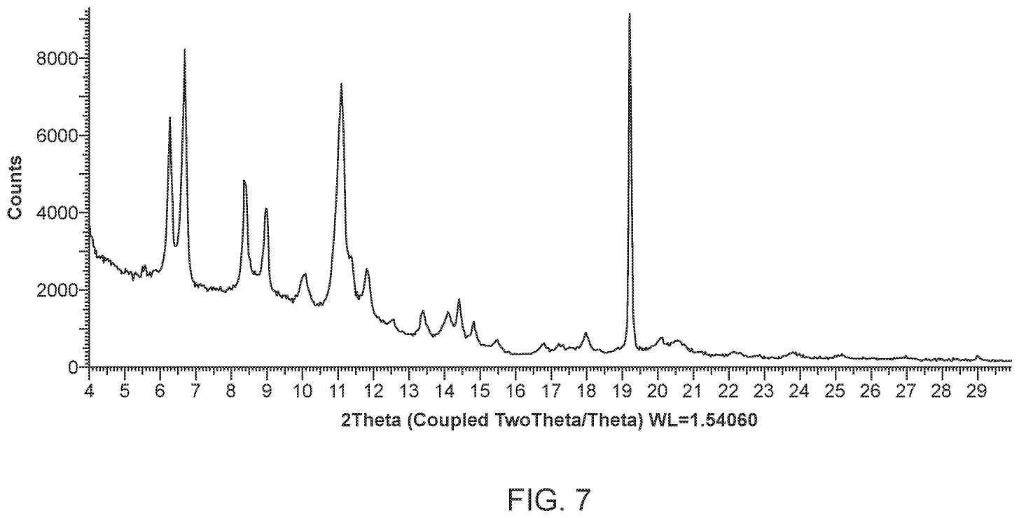

In one embodiment, the crystalline Form C of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 6.2, is characterized by a powder X-ray diffraction pattern that has a characteristic peak in degrees 2θ at about 6.5, is characterized by a powder X-ray diffraction pattern that has a characteristic peak in degrees 2θ at about 6.7, is characterized by a powder X-ray diffraction pattern that has a characteristic peak in degrees 2θ at about 8.4, is characterized by a powder X-ray diffraction pattern that has a characteristic peak in degrees 2θ at about 8.8, is characterized by a powder X-ray diffraction pattern that has a characteristic peak in degrees 2θ at about 11.4, is characterized by a powder X-ray diffraction pattern that has a characteristic peak in degrees 2θ at about 13.5, and/or is characterized by a powder X-ray diffraction pattern that has a characteristic peak in degrees 2θ at about 14.2. In yet another embodiment, crystalline Form C is characterized by a powder X-ray diffraction pattern having at least one or more characteristic peaks in degrees 2θ at about 6.2, 6.5, 6.7, 8.4, 8.8, 10.7, 11.4, 13.5, and 14.2. For example, a contemplated crystalline form has a powder X-ray diffraction pattern shown in FIG. 7. In one embodiment, the powder X-ray diffraction pattern of the crystalline form was obtained using Cu Kα radiation.

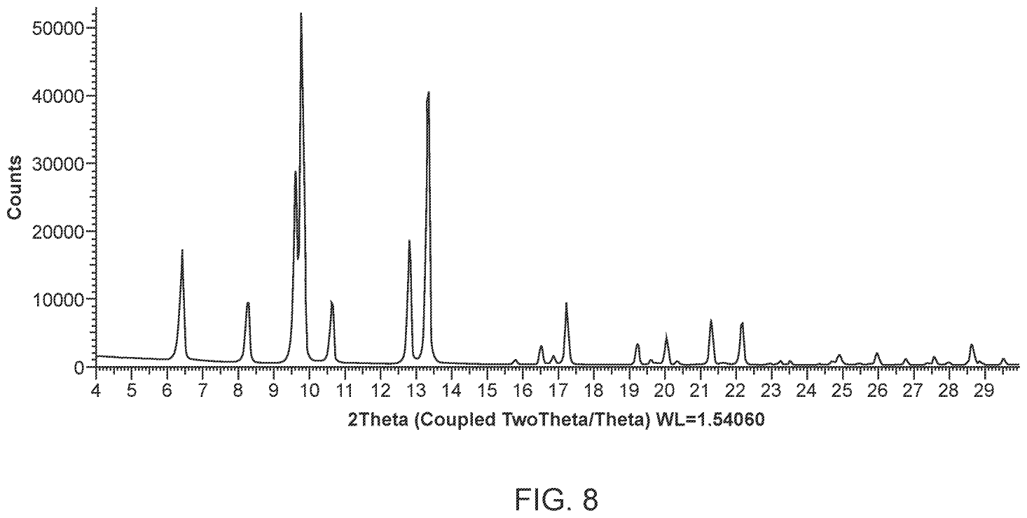

The contemplated crystalline Form C of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a differential scanning calorimetry (DSC) profile showing a characteristic endotherm with an onset of about 92° C. and a peak of about 107° C., a characteristic endotherm with an onset of about 98° C. and a peak of about 119° C., and a characteristic endotherm with an onset of about 161° C. and a peak of about 173° C. Form C, for example, may be characterized by the differential scanning calorimetry profile shown in FIG. 8.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 6.7 (referred to herein as “Form D”), is disclosed herein.

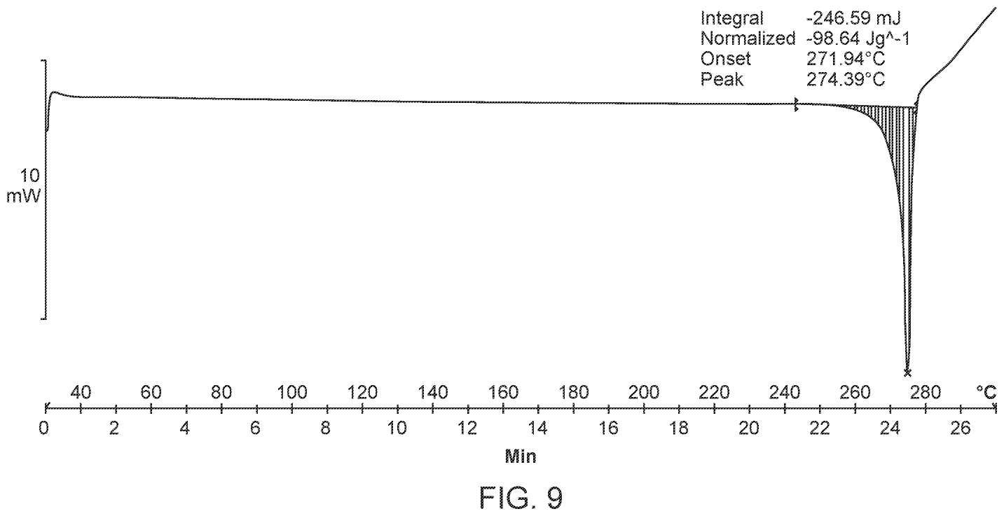

In one embodiment, the crystalline Form D of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 6.3, is characterized by a powder X-ray diffraction pattern that has a characteristic peak in degrees 2θ at about 8.4, is characterized by a powder X-ray diffraction pattern that has a characteristic peak in degrees 2θ at about 9.0, is characterized by a powder X-ray diffraction pattern that has a characteristic peak in degrees 2θ at about 10.0, is characterized by a powder X-ray diffraction pattern that has a characteristic peak in degrees 2θ at about 11.1, is characterized by a powder X-ray diffraction pattern that has a characteristic peak in degrees 2θ at about 11.3, is characterized by a powder X-ray diffraction pattern that has a characteristic peak in degrees 2θ at about 11.8, is characterized by a powder X-ray diffraction pattern that has a characteristic peak in degrees 2θ at about 13.4, is characterized by a powder X-ray diffraction pattern that has a characteristic peak in degrees 2θ at about 14.1, is characterized by a powder X-ray diffraction pattern that has a characteristic peak in degrees 2θ at about 14.4, and/or is characterized by a powder X-ray diffraction pattern that has a characteristic peak in degrees 2θ at about 14.8. In yet another embodiment, crystalline Form D is characterized by a powder X-ray diffraction pattern having at least one or more characteristic peaks in degrees 2θ at about 6.3, 6.7, 8.4, 9.0, 10.0, 11.1, 11.3, 11.8, 13.4, 14.1, 14.4, and 14.8. For example, a contemplated crystalline form has a powder X-ray diffraction pattern shown in FIG. 9. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 9.3 (referred to herein as “Form F”), is disclosed herein.

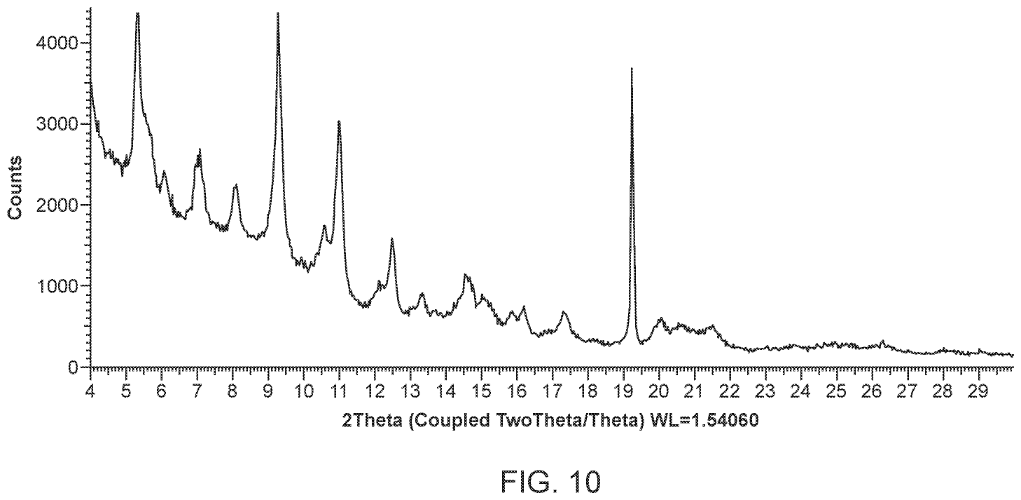

In one embodiment, the crystalline Form F of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 5.3, is characterized by a powder X-ray diffraction pattern that has a characteristic peak in degrees 2θ at about 5.6, is characterized by a powder X-ray diffraction pattern that has a characteristic peak in degrees 2θ at about 6.1, is characterized by a powder X-ray diffraction pattern that has a characteristic peak in degrees 2θ at about 7.0, is characterized by a powder X-ray diffraction pattern that has a characteristic peak in degrees 2θ at about 8.1, is characterized by a powder X-ray diffraction pattern that has a characteristic peak in degrees 2θ at about 10.6, is characterized by a powder X-ray diffraction pattern that has a characteristic peak in degrees 2θ at about 11.0, is characterized by a powder X-ray diffraction pattern that has a characteristic peak in degrees 2θ at about 12.5, is characterized by a powder X-ray diffraction pattern that has a characteristic peak in degrees 2θ at about 14.6, is characterized by a powder X-ray diffraction pattern that has a characteristic peak in degrees 2θ at about 17.3, and/or is characterized by a powder X-ray diffraction pattern that has a characteristic peak in degrees 2θ at about 20.1. In yet another embodiment, crystalline Form F is characterized by a powder X-ray diffraction pattern having at least one or more characteristic peaks in degrees 2θ at about 5.3, 5.6, 6.1, 7.0, 8.1, 9.3, 10.6, 11.0, 12.5, 14.6, 17.3, and 20.1. For example, a contemplated crystalline form has a powder X-ray diffraction pattern shown in FIG. 10. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.4 (referred to herein as “Form G”), is disclosed herein.

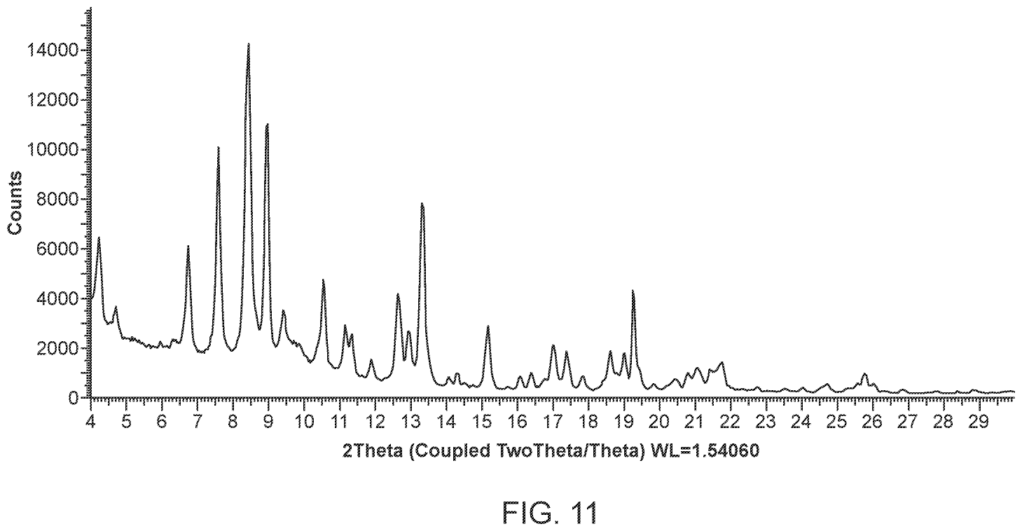

In one embodiment, the crystalline Form G of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 4.2, is characterized by a powder X-ray diffraction pattern that has a characteristic peak in degrees 2θ at about 6.7, is characterized by a powder X-ray diffraction pattern that has a characteristic peak in degrees 2θ at about 7.6, is characterized by a powder X-ray diffraction pattern that has a characteristic peak in degrees 2θ at about 8.9, is characterized by a powder X-ray diffraction pattern that has a characteristic peak in degrees 2θ at about 10.5, is characterized by a powder X-ray diffraction pattern that has a characteristic peak in degrees 2θ at about 12.6, is characterized by a powder X-ray diffraction pattern that has a characteristic peak in degrees 2θ at about 12.9, is characterized by a powder X-ray diffraction pattern that has a characteristic peak in degrees 2θ at about 13.3, is characterized by a powder X-ray diffraction pattern that has a characteristic peak in degrees 2θ at about 15.1, is characterized by a powder X-ray diffraction pattern that has a characteristic peak in degrees 2θ at about 21.4, and/or is characterized by a powder X-ray diffraction pattern that has a characteristic peak in degrees 2θ at about 21.7. In yet another embodiment, crystalline Form G is characterized by a powder X-ray diffraction pattern having at least one or more characteristic peaks in degrees 2θ at about 4.2, 6.7, 7.6, 8.4, 8.9, 10.5, 12.6, 12.9, 13.3, 15.1, 21.4, and 21.7. For example, a contemplated crystalline form has a powder X-ray diffraction pattern shown in FIG. 11. In one embodiment, the powder X-ray diffraction pattern of the crystalline form was obtained using Cu Kα radiation.

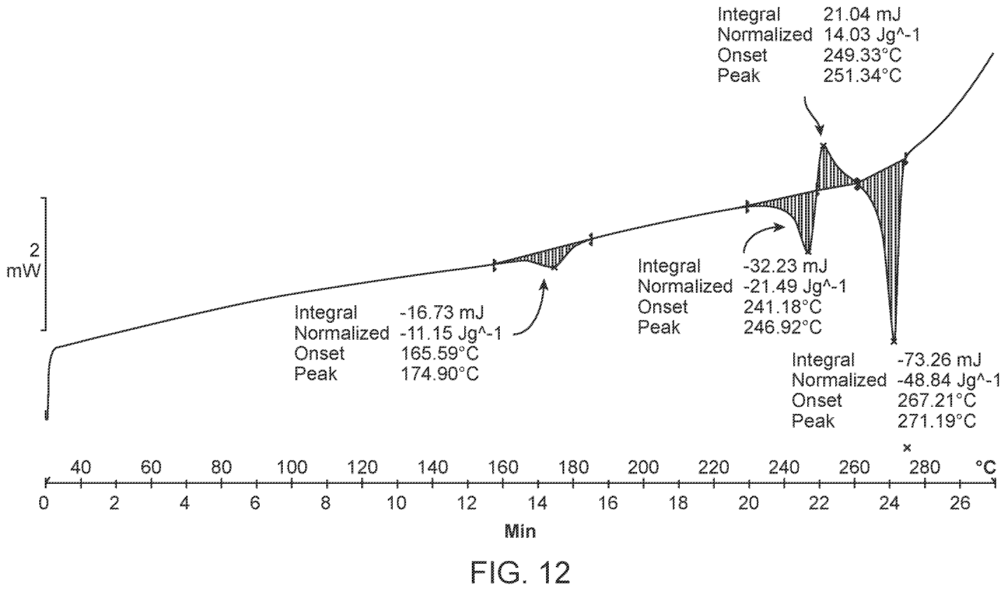

The contemplated crystalline Form G of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a differential scanning calorimetry (DSC) profile showing a characteristic endotherm with an onset of about 166° C. and a peak of about 175° C., a characteristic endotherm with an onset of about 241° C. and a peak of about 245° C., a characteristic exotherm with an onset of about 249° C. and a peak of about 251° C., and a characteristic endotherm with an onset of about 267° C. and a peak of about 271° C. Form G, for example, may be characterized by the differential scanning calorimetry profile shown in FIG. 12.

The contemplated crystalline Form G of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a thermogravimetric analysis (TGA) profile showing a mass loss of about 5.99 wt. % up to about 205° C. In other embodiments, crystalline Form G may be characterized by optical microscopy showing a mixture of irregular and needle-like morphologies.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 9.3 (referred to herein as “Form H”), is disclosed herein.

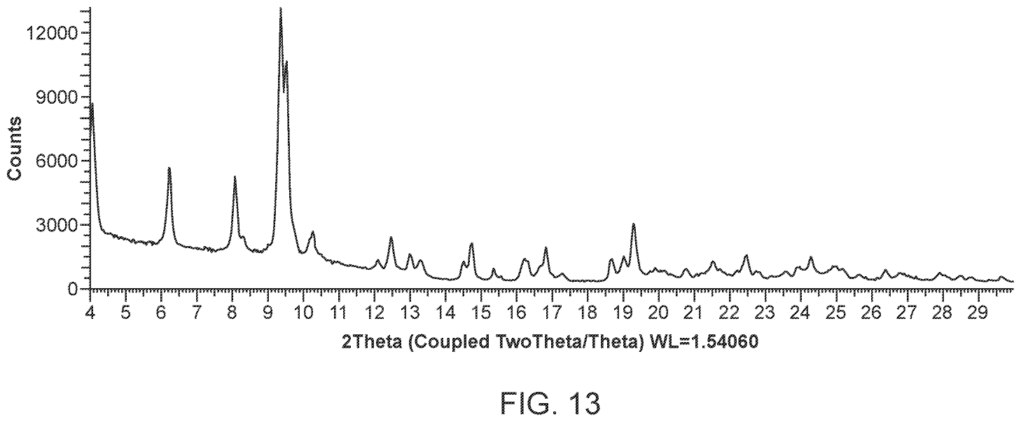

In one embodiment, the crystalline Form H of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 4.1, is characterized by a powder X-ray diffraction pattern that has a characteristic peak in degrees 2θ at about 6.2, is characterized by a powder X-ray diffraction pattern that has a characteristic peak in degrees 2θ at about 8.1, is characterized by a powder X-ray diffraction pattern that has a characteristic peak in degrees 2θ at about 9.5, is characterized by a powder X-ray diffraction pattern that has a characteristic peak in degrees 2θ at about 10.2, is characterized by a powder X-ray diffraction pattern that has a characteristic peak in degrees 2θ at about 12.5, is characterized by a powder X-ray diffraction pattern that has a characteristic peak in degrees 2θ at about 14.7, is characterized by a powder X-ray diffraction pattern that has a characteristic peak in degrees 2θ at about 16.2, is characterized by a powder X-ray diffraction pattern that has a characteristic peak in degrees 2θ at about 16.8, is characterized by a powder X-ray diffraction pattern that has a characteristic peak in degrees 2θ at about 19.0, and/or is characterized by a powder X-ray diffraction pattern that has a characteristic peak in degrees 2θ at about 22.4. In yet another embodiment, crystalline Form H is characterized by a powder X-ray diffraction pattern having at least one or more characteristic peaks in degrees 2θ at about 4.1, 6.2, 8.1, 9.3, 9.5, 10.2, 12.5, 14.7, 16.2, 16.8, 19.0, and 22.4. For example, a contemplated crystalline form has a powder X-ray diffraction pattern shown in FIG. 13. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 10.3 (referred to herein as “Form I”), is disclosed herein.

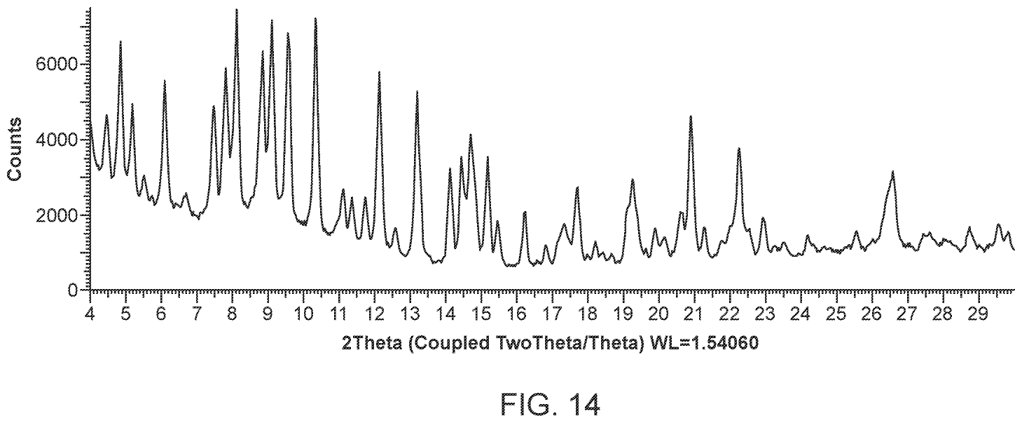

In one embodiment, the crystalline Form I of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 4.8, is characterized by a powder X-ray diffraction pattern that has a characteristic peak in degrees 2θ at about 6.1, is characterized by a powder X-ray diffraction pattern that has a characteristic peak in degrees 2θ at about 7.8, is characterized by a powder X-ray diffraction pattern that has a characteristic peak in degrees 2θ at about 8.1, is characterized by a powder X-ray diffraction pattern that has a characteristic peak in degrees 2θ at about 8.8, is characterized by a powder X-ray diffraction pattern that has a characteristic peak in degrees 2θ at about 9.1, is characterized by a powder X-ray diffraction pattern that has a characteristic peak in degrees 2θ at about 9.6, is characterized by a powder X-ray diffraction pattern that has a characteristic peak in degrees 2θ at about 12.1, is characterized by a powder X-ray diffraction pattern that has a characteristic peak in degrees 2θ at about 13.2, is characterized by a powder X-ray diffraction pattern that has a characteristic peak in degrees 2θ at about 14.7, and/or is characterized by a powder X-ray diffraction pattern that has a characteristic peak in degrees 2θ at about 20.9. In yet another embodiment, crystalline Form I is characterized by a powder X-ray diffraction pattern having at least one or more characteristic peaks in degrees 2θ at about 4.8, 6.1, 7.8, 8.1, 8.8, 9.1, 9.6, 10.3, 12.1, 13.2, 14.7, and 20.9. For example, a contemplated crystalline form has a powder X-ray diffraction pattern shown in FIG. 14. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 10.6 (referred to herein as “Form J”), is disclosed herein.

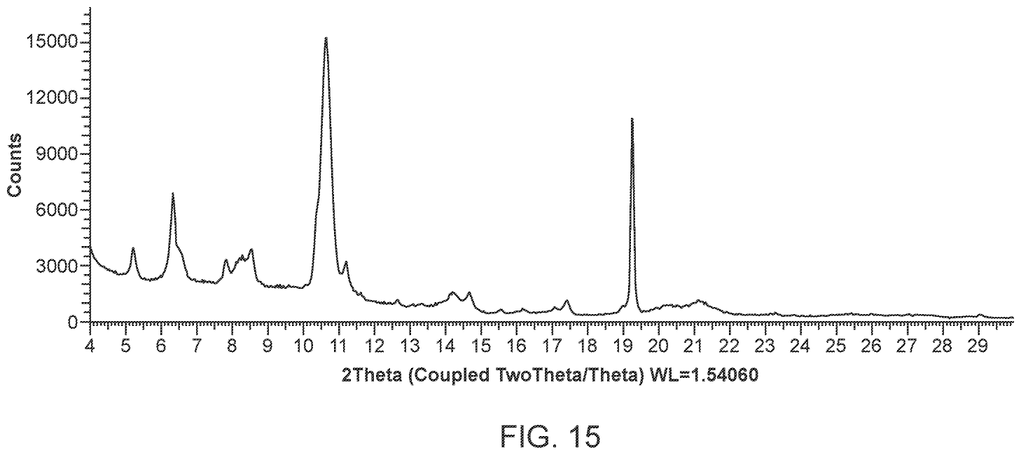

In one embodiment, the crystalline Form J of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 5.2, is characterized by a powder X-ray diffraction pattern that has a characteristic peak in degrees 2θ at about 6.3, is characterized by a powder X-ray diffraction pattern that has a characteristic peak in degrees 2θ at about 6.5, is characterized by a powder X-ray diffraction pattern that has a characteristic peak in degrees 2θ at about 7.8, is characterized by a powder X-ray diffraction pattern that has a characteristic peak in degrees 2θ at about 8.1, is characterized by a powder X-ray diffraction pattern that has a characteristic peak in degrees 2θ at about 8.3, is characterized by a powder X-ray diffraction pattern that has a characteristic peak in degrees 2θ at about 8.5, is characterized by a powder X-ray diffraction pattern that has a characteristic peak in degrees 2θ at about 11.2, is characterized by a powder X-ray diffraction pattern that has a characteristic peak in degrees 2θ at about 14.2, is characterized by a powder X-ray diffraction pattern that has a characteristic peak in degrees 2θ at about 14.7, and/or is characterized by a powder X-ray diffraction pattern that has a characteristic peak in degrees 2θ at about 17.4. In yet another embodiment, crystalline Form J is characterized by a powder X-ray diffraction pattern having at least one or more characteristic peaks in degrees 2θ at about 5.2, 6.3, 6.5, 7.8, 8.1, 8.3, 8.5, 10.6, 11.2, 14.2, 14.7, and 17.4. For example, a contemplated crystalline form has a powder X-ray diffraction pattern shown in FIG. 15. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.7 (referred to herein as “Form K”), is disclosed herein.

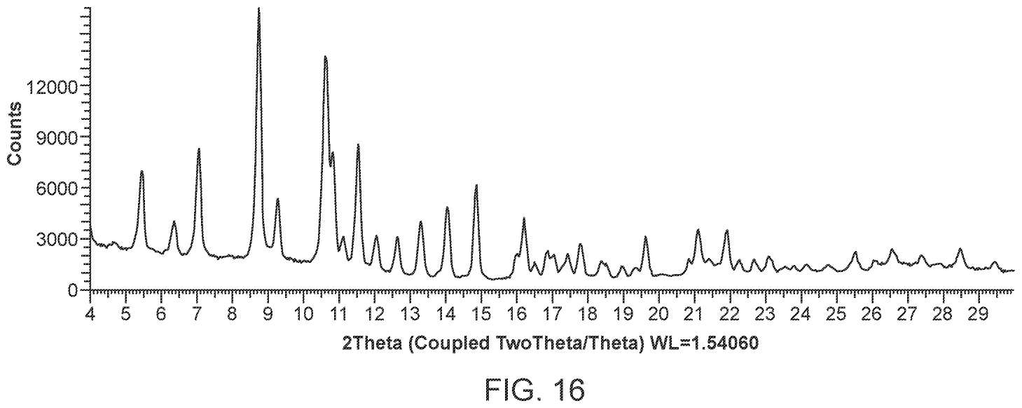

In one embodiment, the crystalline Form K of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 5.5, is characterized by a powder X-ray diffraction pattern that has a characteristic peak in degrees 2θ at about 7.1, is characterized by a powder X-ray diffraction pattern that has a characteristic peak in degrees 2θ at about 9.3, is characterized by a powder X-ray diffraction pattern that has a characteristic peak in degrees 2θ at about 10.6, is characterized by a powder X-ray diffraction pattern that has a characteristic peak in degrees 2θ at about 10.8, is characterized by a powder X-ray diffraction pattern that has a characteristic peak in degrees 2θ at about 11.5, is characterized by a powder X-ray diffraction pattern that has a characteristic peak in degrees 2θ at about 13.3, is characterized by a powder X-ray diffraction pattern that has a characteristic peak in degrees 2θ at about 14.0, is characterized by a powder X-ray diffraction pattern that has a characteristic peak in degrees 2θ at about 14.9, is characterized by a powder X-ray diffraction pattern that has a characteristic peak in degrees 2θ at about 16.2, and/or is characterized by a powder X-ray diffraction pattern that has a characteristic peak in degrees 2θ at about 21.1. In yet another embodiment, crystalline Form K is characterized by a powder X-ray diffraction pattern having at least one or more characteristic peaks in degrees 2θ at about 5.5, 7.1, 8.7, 9.3, 10.6, 10.8, 11.5, 13.3, 14.0, 14.9, 16.2, and 21.1. For example, a contemplated crystalline form has a powder X-ray diffraction pattern shown in FIG. 16. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.9 (referred to herein as “Form U”), is disclosed herein.

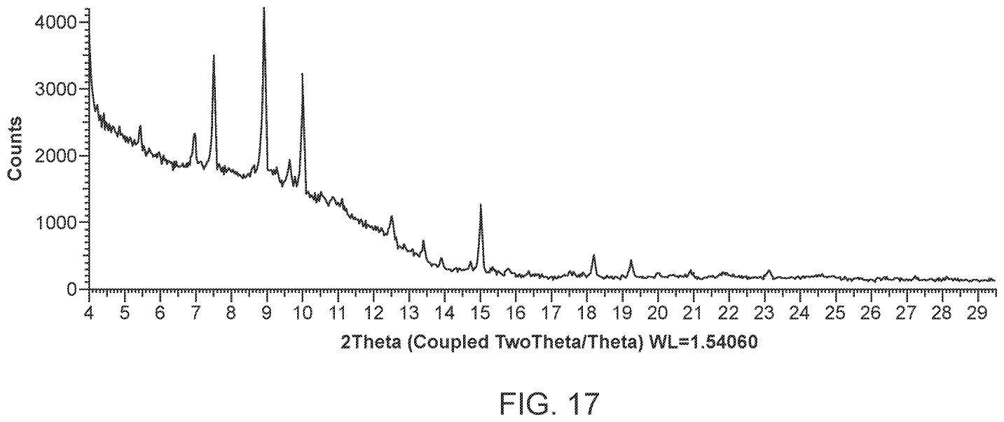

In one embodiment, the crystalline Form L of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 5.4, is characterized by a powder X-ray diffraction pattern that has a characteristic peak in degrees 2θ at about 7.0, is characterized by a powder X-ray diffraction pattern that has a characteristic peak in degrees 2θ at about 7.5, is characterized by a powder X-ray diffraction pattern that has a characteristic peak in degrees 2θ at about 9.1, is characterized by a powder X-ray diffraction pattern that has a characteristic peak in degrees 2θ at about 9.6, is characterized by a powder X-ray diffraction pattern that has a characteristic peak in degrees 2θ at about 10.0, is characterized by a powder X-ray diffraction pattern that has a characteristic peak in degrees 2θ at about 11.1, is characterized by a powder X-ray diffraction pattern that has a characteristic peak in degrees 2θ at about 12.5, is characterized by a powder X-ray diffraction pattern that has a characteristic peak in degrees 2θ at about 15.0, and/or is characterized by a powder X-ray diffraction pattern that has a characteristic peak in degrees 2θ at about 18.2. In yet another embodiment, crystalline Form L is characterized by a powder X-ray diffraction pattern having at least one or more characteristic peaks in degrees 2θ at about 5.4, 7.0, 7.5, 8.9, 9.1, 9.6, 10.0, 11.1, 12.5, 15.0, and 18.2. For example, a contemplated crystalline form has a powder X-ray diffraction pattern shown in FIG. 17. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 7.3 (referred to herein as “Form M”), is disclosed herein.

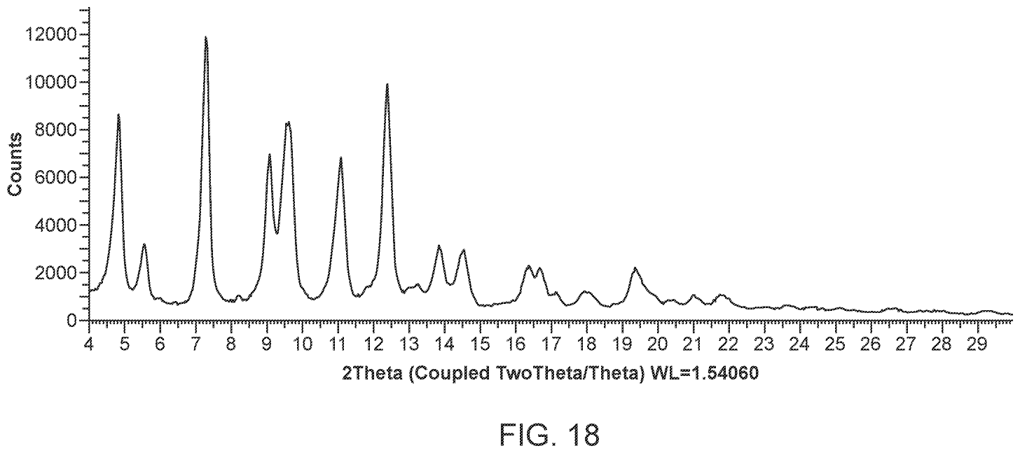

In one embodiment, the crystalline Form M of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 4.8, is characterized by a powder X-ray diffraction pattern that has a characteristic peak in degrees 2θ at about 5.6, is characterized by a powder X-ray diffraction pattern that has a characteristic peak in degrees 2θ at about 9.1, is characterized by a powder X-ray diffraction pattern that has a characteristic peak in degrees 2θ at about 9.6, is characterized by a powder X-ray diffraction pattern that has a characteristic peak in degrees 2θ at about 11.1, is characterized by a powder X-ray diffraction pattern that has a characteristic peak in degrees 2θ at about 12.4, is characterized by a powder X-ray diffraction pattern that has a characteristic peak in degrees 2θ at about 13.8, is characterized by a powder X-ray diffraction pattern that has a characteristic peak in degrees 2θ at about 14.5, is characterized by a powder X-ray diffraction pattern that has a characteristic peak in degrees 2θ at about 16.4, is characterized by a powder X-ray diffraction pattern that has a characteristic peak in degrees 2θ at about 16.7, and/or is characterized by a powder X-ray diffraction pattern that has a characteristic peak in degrees 2θ at about 19.4. In yet another embodiment, crystalline Form M is characterized by a powder X-ray diffraction pattern having at least one or more characteristic peaks in degrees 2θ at about 4.8, 5.6, 7.3, 9.1, 9.6, 11.1, 12.4, 13.8, 14.5, 16.4, 16.7, and 19.4. For example, a contemplated crystalline form has a powder X-ray diffraction pattern shown in FIG. 18. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.9 (referred to herein as “Form N”), is disclosed herein.

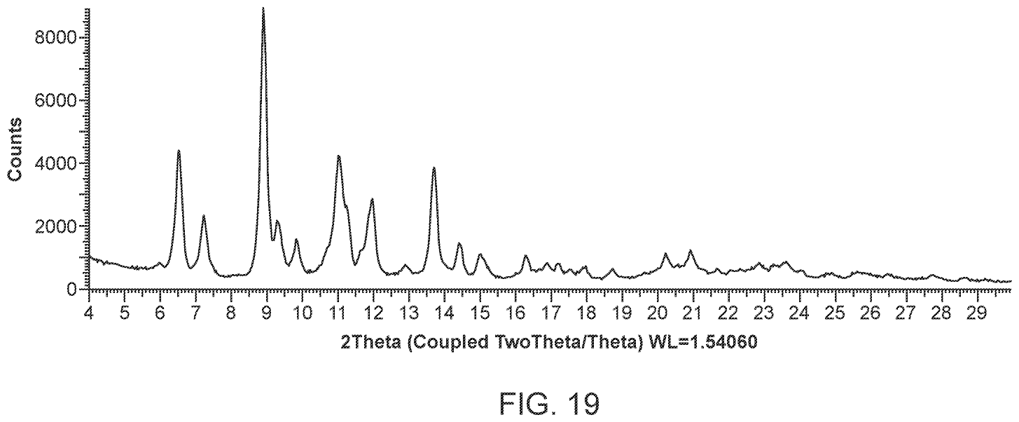

In one embodiment, the crystalline Form N of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 6.5, is characterized by a powder X-ray diffraction pattern that has a characteristic peak in degrees 2θ at about 7.2, is characterized by a powder X-ray diffraction pattern that has a characteristic peak in degrees 2θ at about 9.3, is characterized by a powder X-ray diffraction pattern that has a characteristic peak in degrees 2θ at about 9.8, is characterized by a powder X-ray diffraction pattern that has a characteristic peak in degrees 2θ at about 11.0, is characterized by a powder X-ray diffraction pattern that has a characteristic peak in degrees 2θ at about 12.0, is characterized by a powder X-ray diffraction pattern that has a characteristic peak in degrees 2θ at about 13.7, is characterized by a powder X-ray diffraction pattern that has a characteristic peak in degrees 2θ at about 14.4, is characterized by a powder X-ray diffraction pattern that has a characteristic peak in degrees 2θ at about 15.0, is characterized by a powder X-ray diffraction pattern that has a characteristic peak in degrees 2θ at about 20.2, and/or is characterized by a powder X-ray diffraction pattern that has a characteristic peak in degrees 2θ at about 20.9. In yet another embodiment, crystalline Form N is characterized by a powder X-ray diffraction pattern having at least one or more characteristic peaks in degrees 2θ at about 6.5, 7.2, 8.9, 9.3, 9.8, 11.0, 12.0, 13.7, 14.4, 15.0, 20.2, and 20.9. For example, a contemplated crystalline form has a powder X-ray diffraction pattern shown in FIG. 19. In one embodiment, the powder X-ray diffraction pattern of the crystalline form was obtained using Cu Kα radiation.

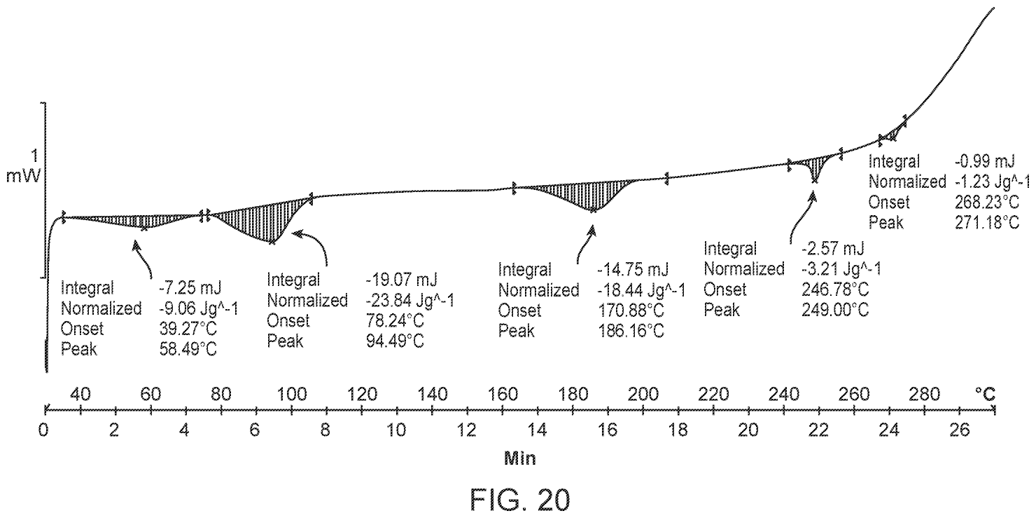

The contemplated crystalline Form N of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a differential scanning calorimetry (DSC) profile showing a characteristic endotherm with an onset of about 39° C. and a peak of about 58° C., a characteristic endotherm with an onset of about 78° C. and a peak of about 94° C., a characteristic endotherm with an onset of about 171° C. and a peak of about 186° C., a characteristic endotherm with an onset of about 245° C. and a peak of about 249° C., and a characteristic endotherm with an onset of about 268° C. and a peak of about 271° C. Form N, for example, may be characterized by the differential scanning calorimetry profile shown in FIG. 20.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.8 (referred to herein as “Form O”), is disclosed herein.

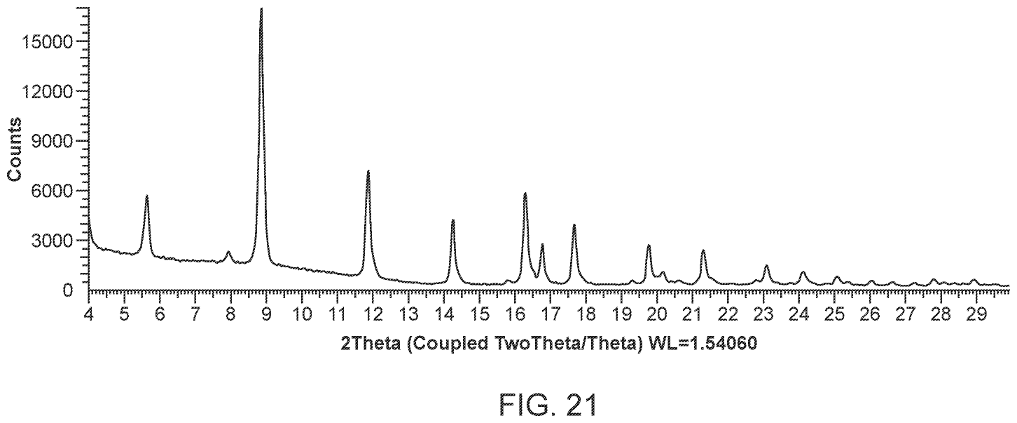

In one embodiment, the crystalline Form O of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 5.6, is characterized by a powder X-ray diffraction pattern that has a characteristic peak in degrees 2θ at about 7.9, is characterized by a powder X-ray diffraction pattern that has a characteristic peak in degrees 2θ at about 11.8, is characterized by a powder X-ray diffraction pattern that has a characteristic peak in degrees 2θ at about 14.2, is characterized by a powder X-ray diffraction pattern that has a characteristic peak in degrees 2θ at about 16.3, is characterized by a powder X-ray diffraction pattern that has a characteristic peak in degrees 2θ at about 16.8, is characterized by a powder X-ray diffraction pattern that has a characteristic peak in degrees 2θ at about 17.7, is characterized by a powder X-ray diffraction pattern that has a characteristic peak in degrees 2θ at about 19.8, is characterized by a powder X-ray diffraction pattern that has a characteristic peak in degrees 2θ at about 20.1, is characterized by a powder X-ray diffraction pattern that has a characteristic peak in degrees 2θ at about 21.3, and/or is characterized by a powder X-ray diffraction pattern that has a characteristic peak in degrees 2θ at about 23.1. In yet another embodiment, crystalline Form O is characterized by a powder X-ray diffraction pattern having at least one or more characteristic peaks in degrees 2θ at about 5.6, 7.9, 8.8, 11.8, 14.2, 16.3, 16.8, 17.7, 19.8, 20.1, 21.3, and 23.1. For example, a contemplated crystalline form has a powder X-ray diffraction pattern shown in FIG. 21. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.9 (referred to herein as “Form P”), is disclosed herein.

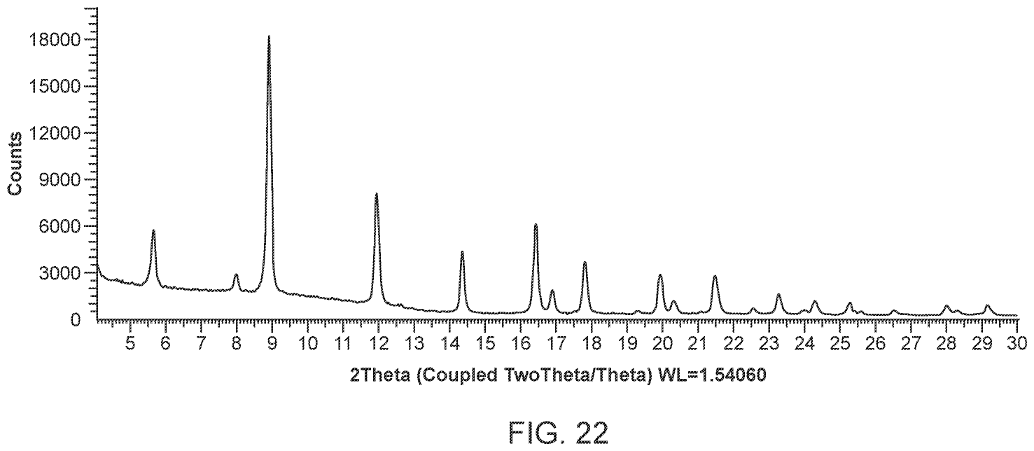

In one embodiment, the crystalline Form P of (S)-12-fluoro-4-(1-methyl-1H-pyrazol-5-yl)-7a,8,13,14-tetrahydro-7H-[1,2,4]triazolo[4′,3′: 1,6]pyrido[3,2-b]benzofuro[4,3-fg][1,4]oxazonine is characterized by a powder X-ray diffraction pattern that has a characteristic peak in degrees 2θ at about 5.6, is characterized by a powder X-ray diffraction pattern that has a characteristic peak in degrees 2θ at about 8.0, is characterized by a powder X-ray diffraction pattern that has a characteristic peak in degrees 2θ at about 12.0, is characterized by a powder X-ray diffraction pattern that has a characteristic peak in degrees 2θ at about 14.4, is characterized by a powder X-ray diffraction pattern that has a characteristic peak in degrees 2θ at about 16.4, is characterized by a powder X-ray diffraction pattern that has a characteristic peak in degrees 2θ at about 16.9, is characterized by a powder X-ray diffraction pattern that has a characteristic peak in degrees 2θ at about 17.8, is characterized by a powder X-ray diffraction pattern that has a characteristic peak in degrees 2θ at about 19.9, is characterized by a powder X-ray diffraction pattern that has a characteristic peak in degrees 2θ at about 20.3, is characterized by a powder X-ray diffraction pattern that has a characteristic peak in degrees 2θ at about 21.5, and/or is characterized by a powder X-ray diffraction pattern that has a characteristic peak in degrees 2θ at about 23.3. In yet another embodiment, crystalline Form P is characterized by a powder X-ray diffraction pattern having at least one or more characteristic peaks in degrees 2θ at about 5.6, 8.0, 8.9, 12.0, 14.4, 16.4, 16.9, 17.8, 19.9, 20.3, 21.5, and 23.3. For example, a contemplated crystalline form has a powder X-ray diffraction pattern shown in FIG. 22. In one embodiment, the powder X-ray diffraction pattern of the crystalline form was obtained using Cu Kα radiation.

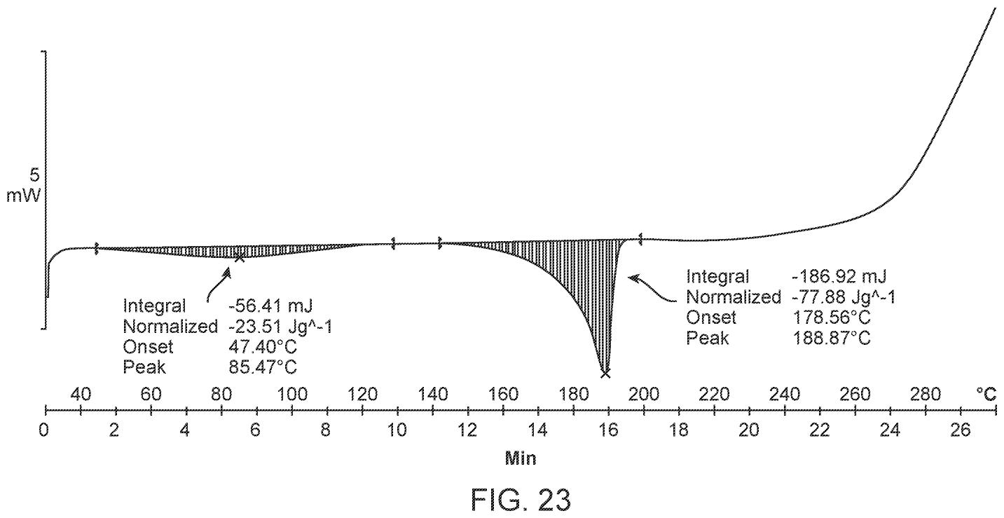

The contemplated crystalline Form P of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a differential scanning calorimetry (DSC) profile showing a characteristic endotherm with an onset of about 47° C. and a peak of about 85° C., and a characteristic endotherm with an onset of about 179° C. and a peak of about 189° C. Form P, for example, may be characterized by the differential scanning calorimetry profile shown in FIG. 23.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.5 (referred to herein as “Form Q”), is disclosed herein.

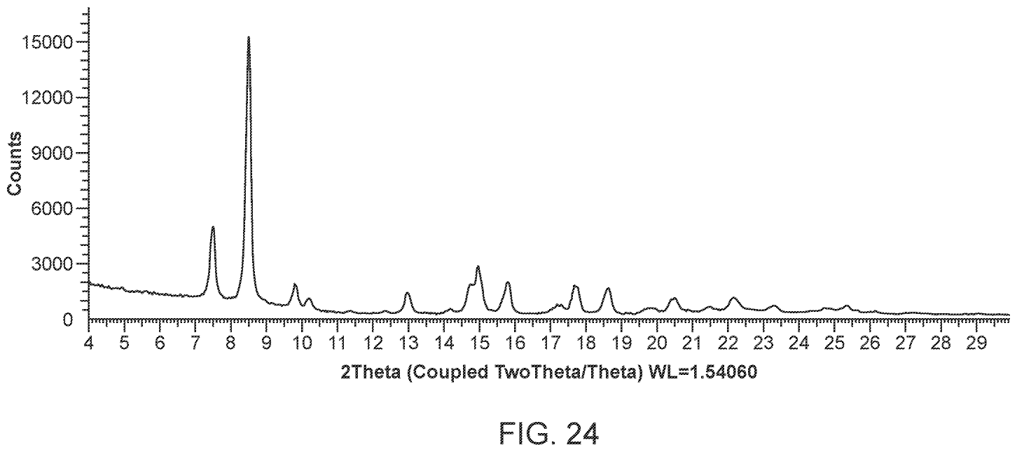

In one embodiment, the crystalline Form Q of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 7.5, is characterized by a powder X-ray diffraction pattern that has a characteristic peak in degrees 2θ at about 9.8, is characterized by a powder X-ray diffraction pattern that has a characteristic peak in degrees 2θ at about 13.0, is characterized by a powder X-ray diffraction pattern that has a characteristic peak in degrees 2θ at about 14.8, is characterized by a powder X-ray diffraction pattern that has a characteristic peak in degrees 2θ at about 15.0, is characterized by a powder X-ray diffraction pattern that has a characteristic peak in degrees 2θ at about 15.8, is characterized by a powder X-ray diffraction pattern that has a characteristic peak in degrees 2θ at about 17.7, is characterized by a powder X-ray diffraction pattern that has a characteristic peak in degrees 2θ at about 18.6, is characterized by a powder X-ray diffraction pattern that has a characteristic peak in degrees 2θ at about 20.5, and/or is characterized by a powder X-ray diffraction pattern that has a characteristic peak in degrees 2θ at about 22.2. In yet another embodiment, crystalline Form Q is characterized by a powder X-ray diffraction pattern having at least one or more characteristic peaks in degrees 2θ at about 7.5, 8.5, 9.8, 13.0, 14.8, 15.0, 15.8, 17.7, 18.6, 20.5, and 22.2. For example, a contemplated crystalline form has a powder X-ray diffraction pattern shown in FIG. 24. In one embodiment, the powder X-ray diffraction pattern of the crystalline form was obtained using Cu Kα radiation.

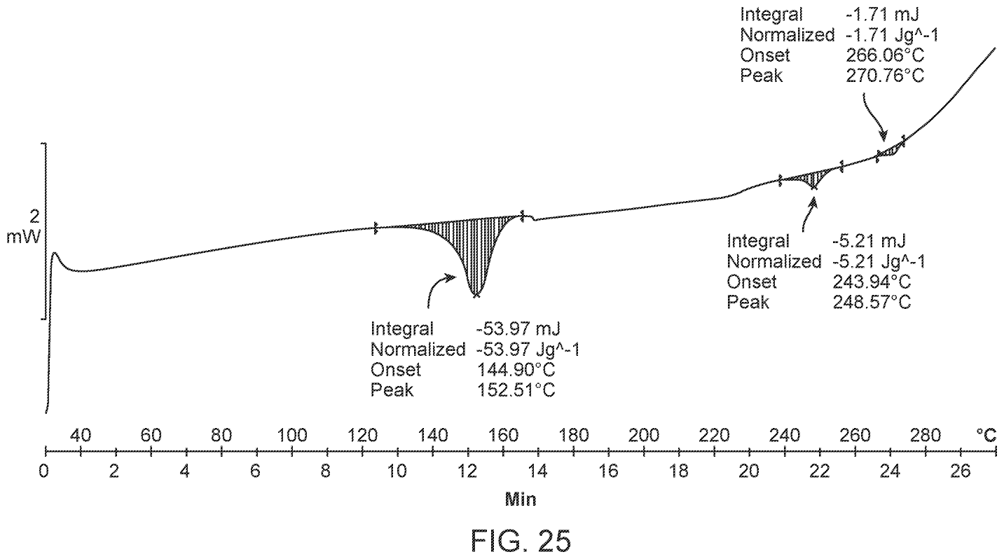

The contemplated crystalline Form Q of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a differential scanning calorimetry (DSC) profile showing a characteristic endotherm with an onset of about 145° C. and a peak of about 152° C., a characteristic endotherm with an onset of about 244° C. and a peak of about 249, and a characteristic endotherm with an onset of about 266° C. and a peak of about 271° C. Form Q, for example, may be characterized by the differential scanning calorimetry profile shown in FIG. 25.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 10.2 (referred to herein as “Form R”), is disclosed herein.

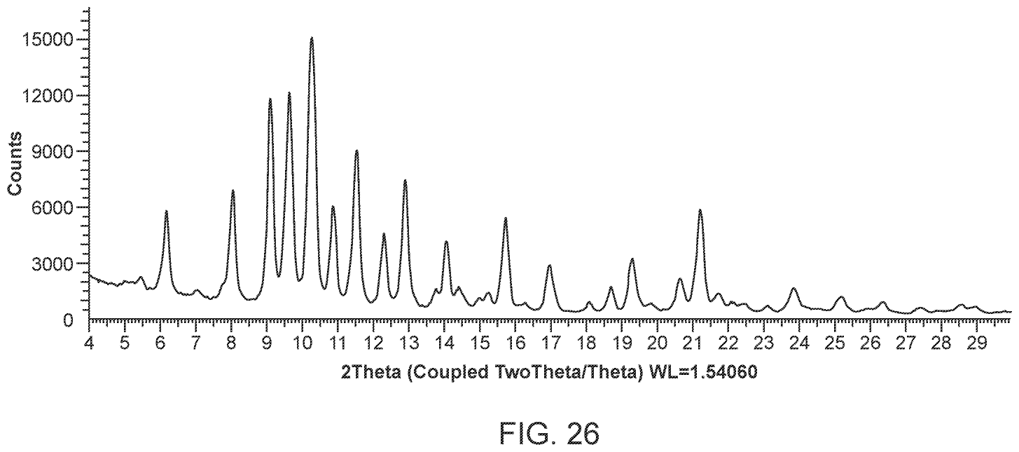

In one embodiment, the crystalline Form R of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 6.2, is characterized by a powder X-ray diffraction pattern that has a characteristic peak in degrees 2θ at about 8.0, is characterized by a powder X-ray diffraction pattern that has a characteristic peak in degrees 2θ at about 9.1, is characterized by a powder X-ray diffraction pattern that has a characteristic peak in degrees 2θ at about 9.6, is characterized by a powder X-ray diffraction pattern that has a characteristic peak in degrees 2θ at about 10.9, is characterized by a powder X-ray diffraction pattern that has a characteristic peak in degrees 2θ at about 11.5, is characterized by a powder X-ray diffraction pattern that has a characteristic peak in degrees 2θ at about 12.3, is characterized by a powder X-ray diffraction pattern that has a characteristic peak in degrees 2θ at about 12.9, is characterized by a powder X-ray diffraction pattern that has a characteristic peak in degrees 2θ at about 14.0, is characterized by a powder X-ray diffraction pattern that has a characteristic peak in degrees 2θ at about 15.7, and/or is characterized by a powder X-ray diffraction pattern that has a characteristic peak in degrees 2θ at about 21.2. In yet another embodiment, crystalline Form R is characterized by a powder X-ray diffraction pattern having at least one or more characteristic peaks in degrees 2θ at about 6.2, 8.0, 9.1, 9.6, 10.2, 10.9, 11.5, 12.3, 12.9, 14.0, 15.7, and 21.2. For example, a contemplated crystalline form has a powder X-ray diffraction pattern shown in FIG. 26. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 9.8 (referred to herein as “Form S”), is disclosed herein.

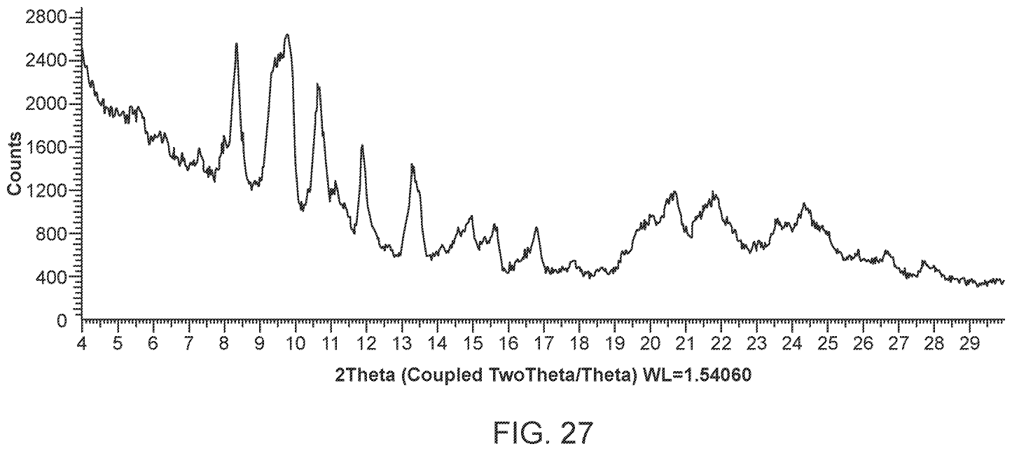

In one embodiment, the crystalline Form S of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 8.3, is characterized by a powder X-ray diffraction pattern that has a characteristic peak in degrees 2θ at about 9.4, is characterized by a powder X-ray diffraction pattern that has a characteristic peak in degrees 2θ at about 10.7, is characterized by a powder X-ray diffraction pattern that has a characteristic peak in degrees 2θ at about 11.9, is characterized by a powder X-ray diffraction pattern that has a characteristic peak in degrees 2θ at about 13.5, is characterized by a powder X-ray diffraction pattern that has a characteristic peak in degrees 2θ at about 15.6, is characterized by a powder X-ray diffraction pattern that has a characteristic peak in degrees 2θ at about 20.1, is characterized by a powder X-ray diffraction pattern that has a characteristic peak in degrees 2θ at about 20.6, is characterized by a powder X-ray diffraction pattern that has a characteristic peak in degrees 2θ at about 21.8, is characterized by a powder X-ray diffraction pattern that has a characteristic peak in degrees 2θ at about 22.2, and/or is characterized by a powder X-ray diffraction pattern that has a characteristic peak in degrees 2θ at about 24.4. In yet another embodiment, crystalline Form S is characterized by a powder X-ray diffraction pattern having at least one or more characteristic peaks in degrees 2θ at about 8.3, 9.4, 9.8, 10.7, 11.9, 13.5, 15.6, 20.1, 20.6, 21.8, 22.2, and 24.4. For example, a contemplated crystalline form has a powder X-ray diffraction pattern shown in FIG. 27. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 6.5 (referred to herein as “Form T”), is disclosed herein.

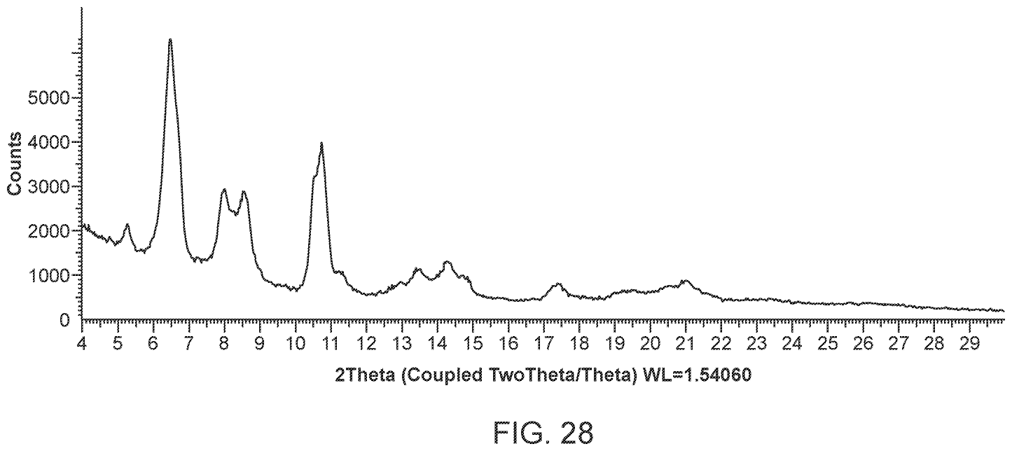

In one embodiment, the crystalline Form T of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 5.3, is characterized by a powder X-ray diffraction pattern that has a characteristic peak in degrees 2θ at about 8.0, is characterized by a powder X-ray diffraction pattern that has a characteristic peak in degrees 2θ at about 8.5, is characterized by a powder X-ray diffraction pattern that has a characteristic peak in degrees 2θ at about 10.7, is characterized by a powder X-ray diffraction pattern that has a characteristic peak in degrees 2θ at about 11.3, is characterized by a powder X-ray diffraction pattern that has a characteristic peak in degrees 2θ at about 13.0, is characterized by a powder X-ray diffraction pattern that has a characteristic peak in degrees 2θ at about 13.5, is characterized by a powder X-ray diffraction pattern that has a characteristic peak in degrees 2θ at about 14.3, is characterized by a powder X-ray diffraction pattern that has a characteristic peak in degrees 2θ at about 14.7, is characterized by a powder X-ray diffraction pattern that has a characteristic peak in degrees 2θ at about 17.4, and/or is characterized by a powder X-ray diffraction pattern that has a characteristic peak in degrees 2θ at about 21.0. In yet another embodiment, crystalline Form T is characterized by a powder X-ray diffraction pattern having at least one or more characteristic peaks in degrees 2θ at about 5.3, 6.5, 8.0, 8.5, 10.7, 11.3, 13.0, 13.5, 14.3, 14.7, 17.4, and 21.0. For example, a contemplated crystalline form has a powder X-ray diffraction pattern shown in FIG. 28. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 9.8 (referred to herein as “Form U”), is disclosed herein.

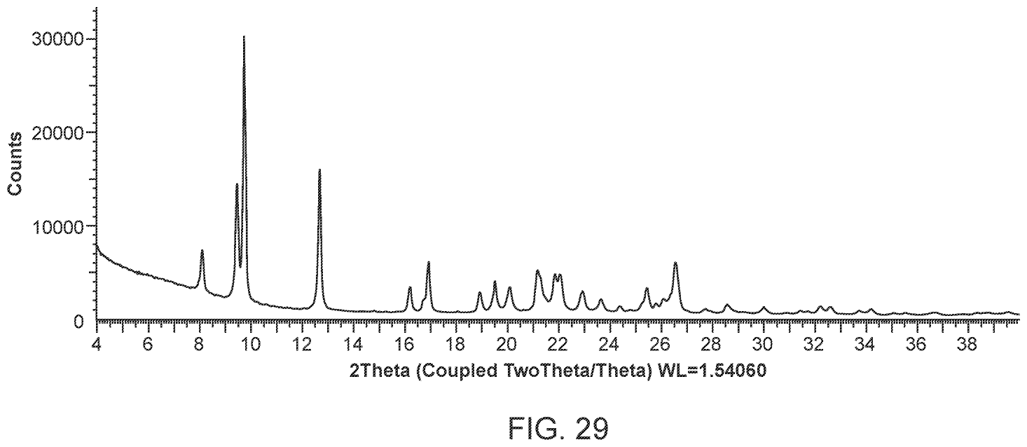

In one embodiment, the crystalline Form U of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 8.1, is characterized by a powder X-ray diffraction pattern that has a characteristic peak in degrees 2θ at about 9.5, is characterized by a powder X-ray diffraction pattern that has a characteristic peak in degrees 2θ at about 12.7, is characterized by a powder X-ray diffraction pattern that has a characteristic peak in degrees 2θ at about 16.2, is characterized by a powder X-ray diffraction pattern that has a characteristic peak in degrees 2θ at about 16.9, is characterized by a powder X-ray diffraction pattern that has a characteristic peak in degrees 2θ at about 18.9, is characterized by a powder X-ray diffraction pattern that has a characteristic peak in degrees 2θ at about 19.5, is characterized by a powder X-ray diffraction pattern that has a characteristic peak in degrees 2θ at about 20.1, is characterized by a powder X-ray diffraction pattern that has a characteristic peak in degrees 2θ at about 21.2, is characterized by a powder X-ray diffraction pattern that has a characteristic peak in degrees 2θ at about 22.0, and/or is characterized by a powder X-ray diffraction pattern that has a characteristic peak in degrees 2θ at about 25.5. In yet another embodiment, crystalline Form U is characterized by a powder X-ray diffraction pattern having at least one or more characteristic peaks in degrees 2θ at about 8.1, 9.5, 9.8, 12.7, 16.2, 16.9, 18.9, 19.5, 20.1, 21.2, 22.0, and 25.5. For example, a contemplated crystalline form has a powder X-ray diffraction pattern shown in FIG. 29. In one embodiment, the powder X-ray diffraction pattern of the crystalline form was obtained using Cu Kα radiation.

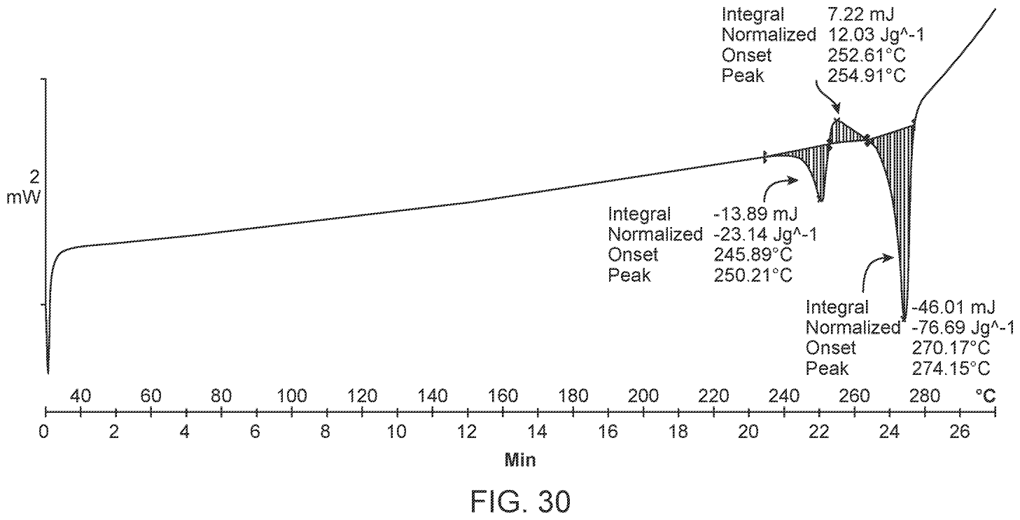

The contemplated crystalline Form U of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a differential scanning calorimetry (DSC) profile showing a characteristic endotherm with an onset of about 246° C. and a peak of about 250° C., a characteristic exotherm with an onset of about 253° C. and a peak of about 255° C., and a characteristic endotherm with an onset of about 270° C. and a peak of about 274° C. Form U, for example, may be characterized by the differential scanning calorimetry profile shown in FIG. 30.

The contemplated crystalline Form U of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a thermogravimetric analysis (TGA) profile showing a mass loss of about 0.007 wt. % up to 255° C. In some embodiments, crystalline Form U may be characterized by a dynamic vapor sorption (DVS) profile showing a mass change of about 0.38 wt. % between about 2 to about 92% relative humidity (RH) at 25° C. In other embodiments, crystalline Form U may be characterized by optical microscopy showing an irregular morphology.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 9.0 (referred to herein as “Form V”), is disclosed herein.

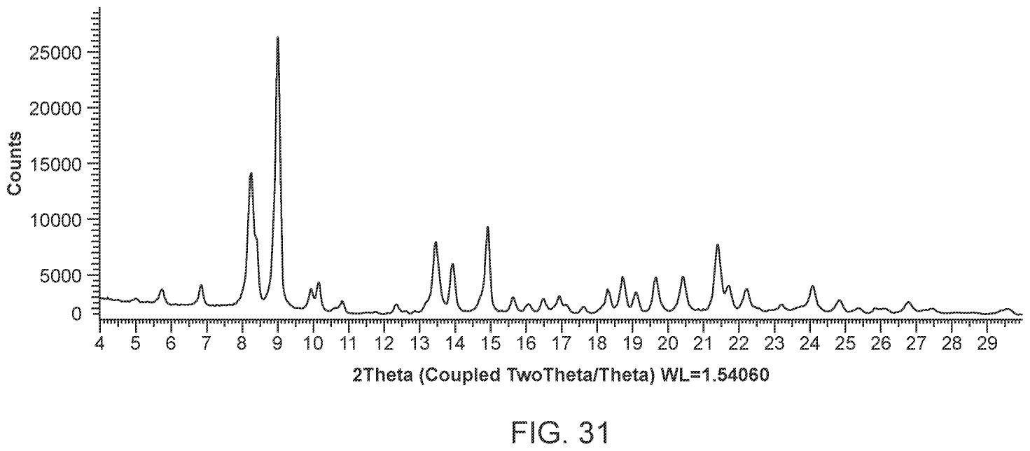

In one embodiment, the crystalline Form V of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 8.2, is characterized by a powder X-ray diffraction pattern that has a characteristic peak in degrees 2θ at about 10.1, is characterized by a powder X-ray diffraction pattern that has a characteristic peak in degrees 2θ at about 13.5, is characterized by a powder X-ray diffraction pattern that has a characteristic peak in degrees 2θ at about 13.9, is characterized by a powder X-ray diffraction pattern that has a characteristic peak in degrees 2θ at about 14.9, is characterized by a powder X-ray diffraction pattern that has a characteristic peak in degrees 2θ at about 18.7, is characterized by a powder X-ray diffraction pattern that has a characteristic peak in degrees 2θ at about 19.6, is characterized by a powder X-ray diffraction pattern that has a characteristic peak in degrees 2θ at about 20.4, is characterized by a powder X-ray diffraction pattern that has a characteristic peak in degrees 2θ at about 21.4, is characterized by a powder X-ray diffraction pattern that has a characteristic peak in degrees 2θ at about 21.7, and/or is characterized by a powder X-ray diffraction pattern that has a characteristic peak in degrees 2θ at about 24.1. In yet another embodiment, crystalline Form V is characterized by a powder X-ray diffraction pattern having at least one or more characteristic peaks in degrees 2θ at about 8.2, 9.0, 10.1, 13.5, 13.9, 14.9, 18.7, 19.6, 20.4, 21.4, 21.7, and 24.1. For example, a contemplated crystalline form has a powder X-ray diffraction pattern shown in FIG. 31. In one embodiment, the powder X-ray diffraction pattern of the crystalline form was obtained using Cu Kα radiation.

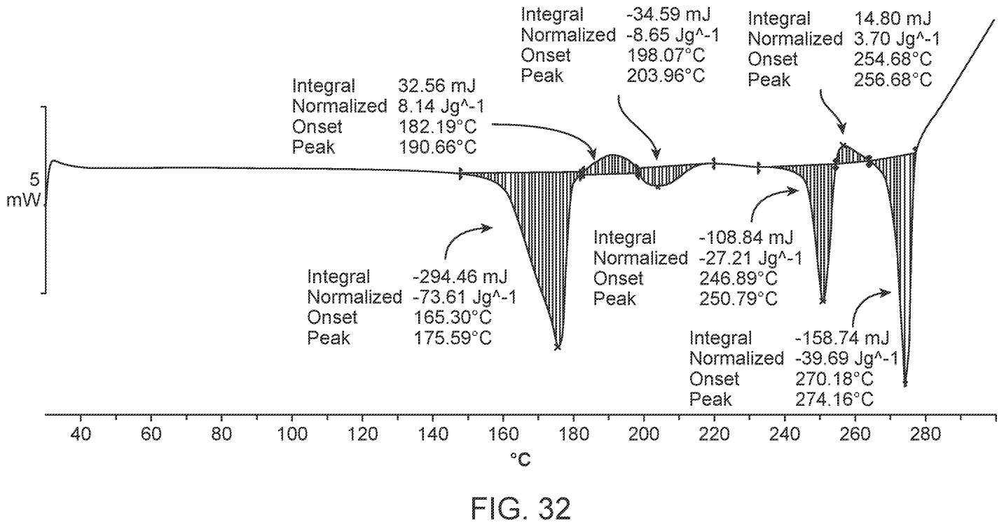

The contemplated crystalline Form V of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a differential scanning calorimetry (DSC) profile showing a characteristic endotherm with an onset of about 165° C. and a peak of about 176° C., a characteristic exotherm with an onset of about 182° C. and a peak of about 191° C., a characteristic endotherm with an onset of about 198° C. and a peak of about 204° C., a characteristic endotherm with an onset of about 247° C. and a peak of about 251° C., a characteristic exotherm with an onset of about 255° C. and a peak of about 257° C., and a characteristic endotherm with an onset of about 270° C. and a peak of about 274° C. Form V, for example, may be characterized by the differential scanning calorimetry profile shown in FIG. 32.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.9 (referred to herein as “Form W”), is disclosed herein.

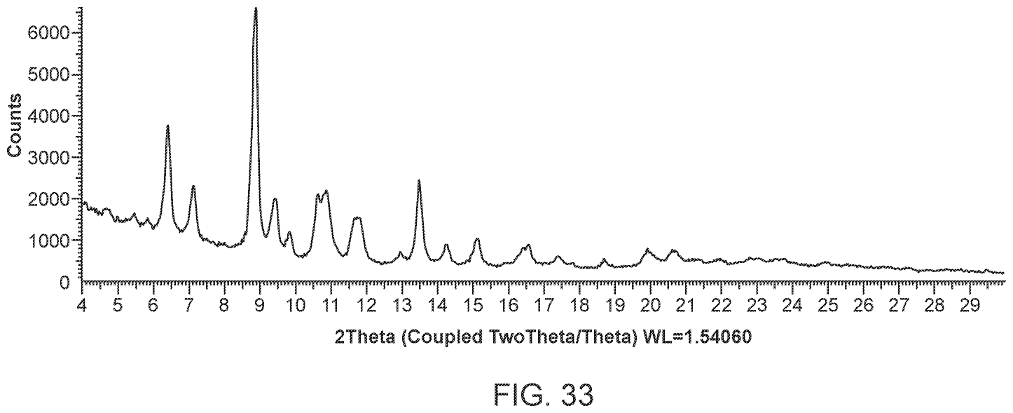

In one embodiment, the crystalline Form W of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 6.4, is characterized by a powder X-ray diffraction pattern that has a characteristic peak in degrees 2θ at about 7.1, is characterized by a powder X-ray diffraction pattern that has a characteristic peak in degrees 2θ at about 9.4, is characterized by a powder X-ray diffraction pattern that has a characteristic peak in degrees 2θ at about 9.8, is characterized by a powder X-ray diffraction pattern that has a characteristic peak in degrees 2θ at about 10.6, is characterized by a powder X-ray diffraction pattern that has a characteristic peak in degrees 2θ at about 10.8, is characterized by a powder X-ray diffraction pattern that has a characteristic peak in degrees 2θ at about 11.8, is characterized by a powder X-ray diffraction pattern that has a characteristic peak in degrees 2θ at about 13.5, is characterized by a powder X-ray diffraction pattern that has a characteristic peak in degrees 2θ at about 14.2, is characterized by a powder X-ray diffraction pattern that has a characteristic peak in degrees 2θ at about 15.1, and/or is characterized by a powder X-ray diffraction pattern that has a characteristic peak in degrees 2θ at about 16.6. In yet another embodiment, crystalline Form W is characterized by a powder X-ray diffraction pattern having at least one or more characteristic peaks in degrees 2θ at about 6.4, 7.1, 8.9, 9.4, 9.8, 10.6, 10.8, 11.8, 13.5, 14.2, 15.1, and 16.6. For example, a contemplated crystalline form has a powder X-ray diffraction pattern shown in FIG. 33. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 9.1 (referred to herein as “Form X”), is disclosed herein.

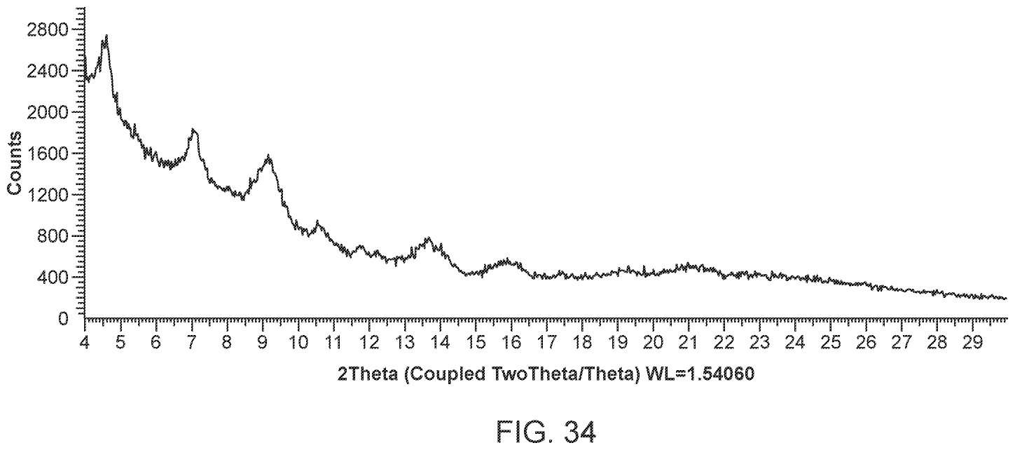

In one embodiment, the crystalline Form X of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 4.5, is characterized by a powder X-ray diffraction pattern that has a characteristic peak in degrees 2θ at about 7.1, is characterized by a powder X-ray diffraction pattern that has a characteristic peak in degrees 2θ at about 10.5, is characterized by a powder X-ray diffraction pattern that has a characteristic peak in degrees 2θ at about 11.8, is characterized by a powder X-ray diffraction pattern that has a characteristic peak in degrees 2θ at about 13.7, is characterized by a powder X-ray diffraction pattern that has a characteristic peak in degrees 2θ at about 14.0, and/or is characterized by a powder X-ray diffraction pattern that has a characteristic peak in degrees 2θ at about 16.1. In yet another embodiment, crystalline Form X is characterized by a powder X-ray diffraction pattern having at least one or more characteristic peaks in degrees 2θ at about 4.5, 7.1, 9.1, 10.5, 11.8, 13.7, 14.0, and 16.1. For example, a contemplated crystalline form has a powder X-ray diffraction pattern shown in FIG. 34. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 10.3 (referred to herein as “Form Y”), is disclosed herein.

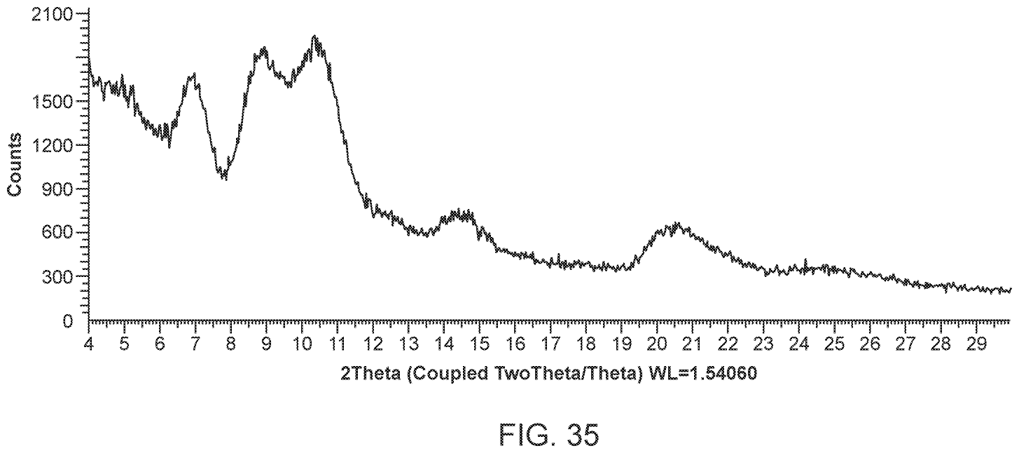

In one embodiment, the crystalline Form Y of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 6.9, is characterized by a powder X-ray diffraction pattern that has a characteristic peak in degrees 2θ at about 8.9, is characterized by a powder X-ray diffraction pattern that has a characteristic peak in degrees 2θ at about 14.4, is characterized by a powder X-ray diffraction pattern that has a characteristic peak in degrees 2θ at about 20.5, and/or is characterized by a powder X-ray diffraction pattern that has a characteristic peak in degrees 2θ at about 20.6. In yet another embodiment, crystalline Form Y is characterized by a powder X-ray diffraction pattern having at least one or more characteristic peaks in degrees 2θ at about 6.9, 8.9, 10.3, 14.4, 20.5, and 20.6. For example, a contemplated crystalline form has a powder X-ray diffraction pattern shown in FIG. 35. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.4 (referred to herein as “Form Z”), is disclosed herein.

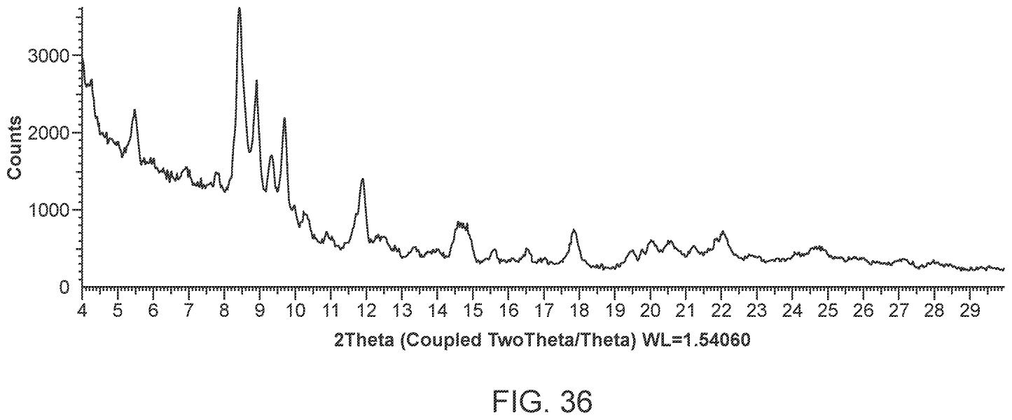

In one embodiment, the crystalline Form Z of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 5.5, is characterized by a powder X-ray diffraction pattern that has a characteristic peak in degrees 2θ at about 8.9, is characterized by a powder X-ray diffraction pattern that has a characteristic peak in degrees 2θ at about 9.3, is characterized by a powder X-ray diffraction pattern that has a characteristic peak in degrees 2θ at about 9.7, is characterized by a powder X-ray diffraction pattern that has a characteristic peak in degrees 2θ at about 11.7, is characterized by a powder X-ray diffraction pattern that has a characteristic peak in degrees 2θ at about 11.9, is characterized by a powder X-ray diffraction pattern that has a characteristic peak in degrees 2θ at about 14.7, is characterized by a powder X-ray diffraction pattern that has a characteristic peak in degrees 2θ at about 14.8, is characterized by a powder X-ray diffraction pattern that has a characteristic peak in degrees 2θ at about 17.9, is characterized by a powder X-ray diffraction pattern that has a characteristic peak in degrees 2θ at about 20.1, and/or is characterized by a powder X-ray diffraction pattern that has a characteristic peak in degrees 2θ at about 22.0. In yet another embodiment, crystalline Form Z is characterized by a powder X-ray diffraction pattern having at least one or more characteristic peaks in degrees 2θ at about 5.5, 8.4, 8.9, 9.3. 9.7, 11.7, 11.9, 14.7, 14.8, 17.9, 20.1, and 22.0. For example, a contemplated crystalline form has a powder X-ray diffraction pattern shown in FIG. 36. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 9.6 (referred to herein as “Form α”), is disclosed herein.

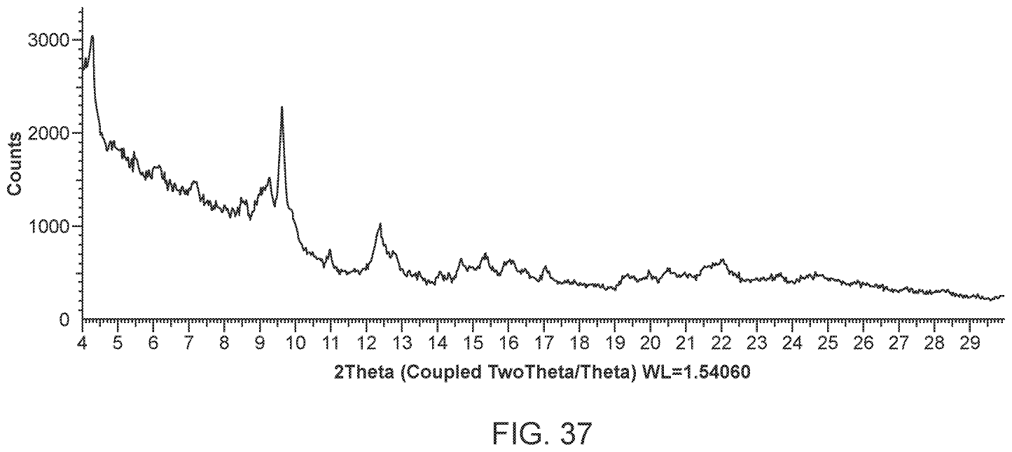

In one embodiment, the crystalline Form α of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 4.3, is characterized by a powder X-ray diffraction pattern that has a characteristic peak in degrees 2θ at about 8.5, is characterized by a powder X-ray diffraction pattern that has a characteristic peak in degrees 2θ at about 9.2, is characterized by a powder X-ray diffraction pattern that has a characteristic peak in degrees 2θ at about 9.3, is characterized by a powder X-ray diffraction pattern that has a characteristic peak in degrees 2θ at about 9.7, is characterized by a powder X-ray diffraction pattern that has a characteristic peak in degrees 2θ at about 12.4, is characterized by a powder X-ray diffraction pattern that has a characteristic peak in degrees 2θ at about 12.8, is characterized by a powder X-ray diffraction pattern that has a characteristic peak in degrees 2θ at about 14.7, is characterized by a powder X-ray diffraction pattern that has a characteristic peak in degrees 2θ at about 15.4, is characterized by a powder X-ray diffraction pattern that has a characteristic peak in degrees 2θ at about 16.0, and/or is characterized by a powder X-ray diffraction pattern that has a characteristic peak in degrees 2θ at about 17.0. In yet another embodiment, crystalline Form α is characterized by a powder X-ray diffraction pattern having at least one or more characteristic peaks in degrees 2θ at about 4.3, 8.5, 9.2, 9.3, 9.6, 9.7, 12.4, 12.8, 14.7, 15.4, 16.0, and 17.0. For example, a contemplated crystalline form has a powder X-ray diffraction pattern shown in FIG. 37. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.9 (referred to herein as “Form J”), is disclosed herein.

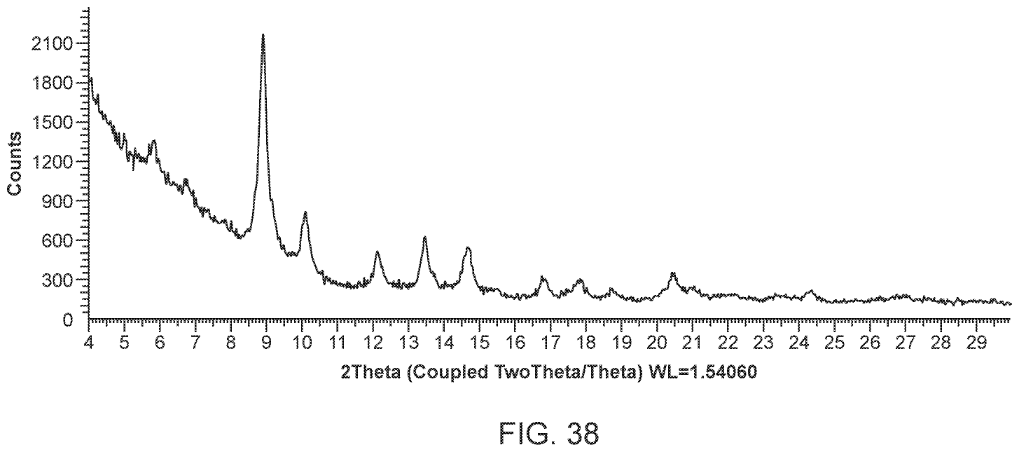

In one embodiment, the crystalline Form β of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 5.8, is characterized by a powder X-ray diffraction pattern that has a characteristic peak in degrees 2θ at about 6.7, is characterized by a powder X-ray diffraction pattern that has a characteristic peak in degrees 2θ at about 10.1, is characterized by a powder X-ray diffraction pattern that has a characteristic peak in degrees 2θ at about 12.1, is characterized by a powder X-ray diffraction pattern that has a characteristic peak in degrees 2θ at about 13.4, is characterized by a powder X-ray diffraction pattern that has a characteristic peak in degrees 2θ at about 14.7, is characterized by a powder X-ray diffraction pattern that has a characteristic peak in degrees 2θ at about 16.8, is characterized by a powder X-ray diffraction pattern that has a characteristic peak in degrees 2θ at about 17.8, is characterized by a powder X-ray diffraction pattern that has a characteristic peak in degrees 2θ at about 20.5, is characterized by a powder X-ray diffraction pattern that has a characteristic peak in degrees 2θ at about 21.0, and/or is characterized by a powder X-ray diffraction pattern that has a characteristic peak in degrees 2θ at about 24.3. In yet another embodiment, crystalline Form β is characterized by a powder X-ray diffraction pattern having at least one or more characteristic peaks in degrees 2θ at about 5.8, 6.7, 8.9, 10.1, 12.1, 13.4, 14.7, 16.8, 17.8, 20.5, 21.0, and 24.3. For example, a contemplated crystalline form has a powder X-ray diffraction pattern shown in FIG. 38. In one embodiment, the powder X-ray diffraction pattern of the crystalline form was obtained using Cu Kα radiation.

In another embodiment, a different crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, characterized by a powder X-ray diffraction pattern having a characteristic peak in degrees 2θ at about 8.9 (referred to herein as “Form γ”), is disclosed herein.

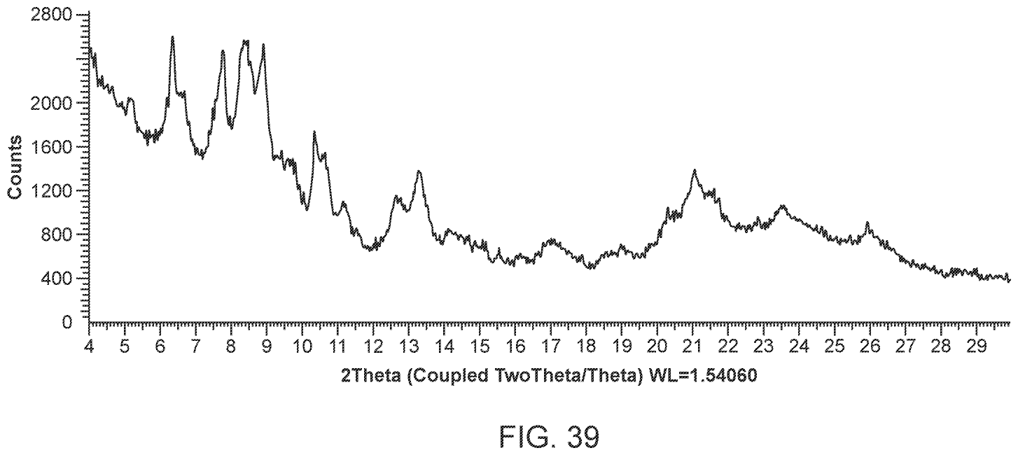

In one embodiment, the crystalline Form γ of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is characterized by a powder X-ray diffraction pattern that has a characteristic peak in degrees 2θ at about 6.3, is characterized by a powder X-ray diffraction pattern that has a characteristic peak in degrees 2θ at about 6.6, is characterized by a powder X-ray diffraction pattern that has a characteristic peak in degrees 2θ at about 7.8, is characterized by a powder X-ray diffraction pattern that has a characteristic peak in degrees 2θ at about 8.4, is characterized by a powder X-ray diffraction pattern that has a characteristic peak in degrees 2θ at about 10.4, is characterized by a powder X-ray diffraction pattern that has a characteristic peak in degrees 2θ at about 10.6, is characterized by a powder X-ray diffraction pattern that has a characteristic peak in degrees 2θ at about 12.6, is characterized by a powder X-ray diffraction pattern that has a characteristic peak in degrees 2θ at about 13.3, is characterized by a powder X-ray diffraction pattern that has a characteristic peak in degrees 2θ at about 20.3, is characterized by a powder X-ray diffraction pattern that has a characteristic peak in degrees 2θ at about 21.1, and/or is characterized by a powder X-ray diffraction pattern that has a characteristic peak in degrees 2θ at about 21.7. In yet another embodiment, crystalline Form γ is characterized by a powder X-ray diffraction pattern having at least one or more characteristic peaks in degrees 2θ at about 6.3, 6.6, 7.8, 8.4, 8.9, 10.4, 10.6, 12.6, 13.3, 20.3, 21.1, and 21.7. For example, a contemplated crystalline form has a powder X-ray diffraction pattern shown in FIG. 39. In one embodiment, the powder X-ray diffraction pattern of the crystalline form was obtained using Cu Kα radiation.

In another embodiment, a substantially amorphous form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is disclosed herein.

In a further embodiment, a pharmaceutical composition comprising a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine and a pharmaceutically acceptable excipient is disclosed herein. For example, a pharmaceutical composition formed from a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is disclosed herein. For example, a pharmaceutical composition comprising the crystalline Form E of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine and a pharmaceutically acceptable excipient is disclosed herein. For example, a pharmaceutical composition formed from the crystalline Form E of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is disclosed herein. In some embodiments, a disclosed pharmaceutical composition may be a formulation for oral administration.

In yet another embodiment, a pharmaceutical composition comprising a disclosed amorphous form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine and a pharmaceutically acceptable excipient is disclosed herein.

In an embodiment, a drug substance comprising at least a detectable amount of a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is disclosed herein. In another embodiment, a drug substance comprising a substantially pure crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is disclosed herein. For example, a drug substance comprising a substantially pure crystalline Form E of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is disclosed herein.

### Compositions

Another aspect of the disclosure provides pharmaceutical compositions comprising crystalline compounds as disclosed herein formulated together with a pharmaceutically acceptable excipient. In particular, the present disclosure provides pharmaceutical compositions comprising crystalline compounds as disclosed herein formulated together with one or more pharmaceutically acceptable excipients. These formulations include those suitable for oral, topical (e.g., transdermal), buccal, ocular, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral, subcutaneous or intravenous administration.

Exemplary pharmaceutical compositions of this disclosure may be used in the form of a pharmaceutical preparation, for example, in solid, semisolid or liquid form, which contains one or more of the compound of the disclosure, as an active ingredient, in admixture with an organic or inorganic excipient or excipient suitable for external, enteral or parenteral applications. The active ingredient may be compounded, for example, with the usual non-toxic, pharmaceutically acceptable excipients for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical excipient, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable excipients, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, nano-suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or excipients comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition includes powders, sprays, ointments, pastes, creams, lotions, gels, solutions, patches and inhalants. The active component may be mixed under sterile conditions with a pharmaceutically acceptable excipient, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable excipients and stabilizers. The excipients and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous excipients which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants. For example, crystalline forms provided herein may be milled to obtain a particular particle size, and in at least some embodiments, such crystalline forms may remain substantially stable upon milling.

Amounts of a crystalline compound as described herein in a formulation may vary according to factors such as the disease state, age, sex, and weight of the individual. Dosage regimens can be adjusted to provide the optimum therapeutic response. For example, a single bolus can be administered, several divided doses may be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active crystalline compound calculated to produce the desired therapeutic effect in association with the required pharmaceutical carrier.

The specification for the dosage unit forms of the disclosure are dictated by and directly dependent on (a) the unique characteristics of the crystalline compound selected and the particular therapeutic effect to be achieved, and (b) the limitations inherent in the art of compounding such an active crystalline compound for the treatment of sensitivity in individuals.

Disclosed compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is suitable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin.

A disclosed crystalline compound can be administered in a time release formulation, for example in a composition which includes a slow release polymer. The crystalline compound can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

In accordance with an alternative aspect of the disclosure, a disclosed crystalline compound can be formulated with one or more additional compounds that enhance the solubility of the compound.

### Methods

In some embodiments, the disclosure provides a method of treating a disease or disorder associated with modulation of Embryonic Ectoderm Development (EED) in a patient in need thereof, comprising administering to the patient an effective amount of a disclosed crystalline compound, for example, a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine. In other embodiments, the disclosure provides a method of treating a disease or disorder associated with modulation of Embryonic Ectoderm Development (EED) in a patient in need thereof, comprising administering to the patient an effective amount of a pharmaceutical composition comprising a disclosed crystalline compound, for example, a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

In some embodiments, the disclosure provides a method of treating a disease or disorder associated with modulation of Polycomb Repressive Complex 2 (PRC2) in a patient in need thereof, comprising administering to the patient an effective amount of a disclosed crystalline compound, for example, a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine. In other embodiments, the disclosure provides a method of treating a disease or disorder associated with modulation of Polycomb Repressive Complex 2 (PRC2) in a patient in need thereof, comprising administering to the patient an effective amount of a pharmaceutical composition comprising a disclosed crystalline compound, for example, a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

In some embodiments, the disease or disorder may be a blood disorder. In certain embodiments, the disclosure provides a method of treating a blood disorder in a patient in need thereof, comprising administering to the patient an effective amount of a disclosed crystalline compound, for example, a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine. In other embodiments, the disclosure provides a method of treating a blood disorder in a patient in need thereof, comprising administering to the patient an effective amount of a pharmaceutical composition comprising a disclosed crystalline compound, for example, a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

In certain embodiments, the blood disorder may be selected from the group consisting of, for example, acute lymphoblastic leukemia (ALL), acute myeloid leukemia (ANIL), amyloidosis, anemia, aplastic anemia, bone marrow failure syndromes, chronic lymphocytic leukemia (CLL), chronic myeloid leukemia (CML), deep vein thrombosis (DVT), Diamond-Blackfan anemia, diffused large B cell lymphoma, dyskeratosis congenita (DKC), eosinophilic disorder, essential thrombocythemia, Fanconi anemia, follicular lymphoma, Gaucher disease, hemochromatosis, hemolytic anemia, hemophilia, hereditary spherocytosis, Hodgkin's lymphoma, idiopathic thrombocytopenic purpura (ITP), inherited bone marrow failure syndromes, iron-deficiency anemia, Langerhans ceil histiocytosis, large granular lymphocytic (LGL) leukemia, leukemia, leukopenia, mastocytosis, monoclonal gammopathy, multiple myeloma, myelodysplastic syndromes (MDS), myelofibrosis, myeloproliferative neoplasms (MPN), non-Hodgkin's lymphoma, paroxysmal nocturnal hemoglobinuria (PNH), pernicious anemia (B12 deficiency), polycythemia vera, porphyria, post-transplant lymphoproliferative disorder (PTLD), pulmonary embolism (PE), Shwachman-Diamond syndrome (SDS), sickle cell disease (SCD), β-thalassemia, thrombocytopenia, thrombotic thrombocytopenic purpura (TTP), venous thromboembolism, Von Willebrand disease, and Valdenstrom's macroglobulinemia (lymphoplasmacytic lymphoma). In some embodiments, the blood disorder is sickle cell disease (SCD). In other embodiments, the blood disorder is β-thalassemia.

In some embodiments, the disease or disorder may be a cancer. In certain embodiments, the disclosure provides a method of treating a cancer in a patient in need thereof, comprising administering to the patient an effective amount of a disclosed crystalline compound, for example, a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine. In other embodiments, the disclosure provides a method of treating a cancer in a patient in need thereof, comprising administering to the patient an effective amount of a pharmaceutical composition comprising a disclosed crystalline compound, for example, a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

In certain embodiments, the cancer may be selected from the group consisting of, for example, mesothelioma, gastric cancer, malignant rhabdoid tumor, hepatocellular carcinoma, prostate cancer, breast carcinoma, bile duct and gallbladder cancers, bladder carcinoma, brain tumors including neuroblastoma, Schwannoma, glioma, glioblastoma and astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancers, parathyroid tumors, uterine tumors, and soft tissue sarcomas.

In other embodiments, the disclosure provides a method of treating thoracic aortic aneurysm, coronary heart disease, stenotic disease, pulmonary artery hypertension (PAR), liver fibrosis, allergic inflammation, retinitis pigmentosa, septic shock, herpes simplex virus, human cytomegalovirus, α-thalassemia, familial atrial fibrillation, common variable immunodeficiency, aneurysm-osteoarthritis syndrome, and acquired immunodeficiency syndrome in a patient in need thereof, comprising administering to the patient an effective amount of a disclosed crystalline compound, for example, a disclosed crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

In other embodiments, the disclosure provides a method of treating thoracic aortic aneurysm, coronary heart disease, stenotic disease, pulmonary artery hypertension (PAR), liver fibrosis, allergic inflammation, retinitis pigmentosa, septic shock, herpes simplex virus, human cytomegalovirus, α-thalassemia, familial atrial fibrillation, common variable immunodeficiency, aneurysm-osteoarthritis syndrome, and acquired immunodeficiency syndrome in a patient in need thereof, comprising administering to the patient an effective amount of a pharmaceutical composition comprising a disclosed crystalline compound, for example, a disclosed crystalline form of (S)-12-fluoro-4-(1-methyl-1H-pyrazol-5-yl)-7a,8,13,14-tetrahydro-7H-[1,2,4]triazolo[4′,3′: 1,6]pyrido[3,2-b]benzofuro[4,3-fg][1,4]oxazonine.

In particular, in certain embodiments, the disclosure provides a method of treating the above medical indications comprising administering to a patient in need thereof an effective amount of a crystalline compound disclosed herein. In certain other embodiments, the disclosure provides a method of treating the above medical conditions in a patient in need thereof, comprising orally, subcutaneously, or intravenously administering to the patient a composition comprising a disclosed crystalline form.

The crystalline compounds disclosed herein can be used as a medicament or pharmaceutically acceptable composition, e.g., in the form of pharmaceutical preparations for oral, enteral, parenteral, or topical administration, and the contemplated methods disclosed herein may include administering orally, enterally, parenterally, or topically a disclosed crystalline compound, or a composition comprising or formed from such a disclosed crystalline compound. For example, a disclosed crystalline form may be capable of controlling one or more pharmacokinetic properties (e.g., a longer or shorter release profile) when administered by a certain route (e.g., oral) or in a certain formulation, as compared to a different route (e.g., subcutaneous) or other formulation e.g., a formulation having the amorphous form. In one embodiment, a disclosed crystalline form may afford substantial reproducibility from one formulation to another.

Also disclosed herein are pharmaceutical compositions comprising a disclosed crystalline compound and at least one additional therapeutic agent. In some embodiments, the additional therapeutic agent may be selected from the group consisting of, for example, anti-cancer agents, immunomodulators, anti-allergic agents, anti-emetics, pain relievers, cytoprotective agents, anti-sickling agents, and combinations thereof. In other embodiments, the additional therapeutic agent may be, for example, an EZH2 inhibitor. For example, in certain embodiments the additional therapeutic agent may be selected from the group consisting of N-((4,6-dimethyl-2-oxo-1,2-dihydropyridin-3-yl)methyl)-5-(ethyl(tetrahydro-2H-pyran-4-yl)amino)-4-methyl-4′-(morpholinomethyl)-[1,1′-biphenyl]-3-carboxamide (tazemetostat), (2R)-7-chloro-2-[4-(dirnethylamino)cyclohexyl]-N-[(4,6-dimethyl-2-oxo-1H-pyridin-3-yl)methyl]-2,4-dimethyl-1,3-benzodioxole-5-carboxamide (valemetostat, DS-3201b), N-[(4-methoxy-6-methyl-2-oxo-1H-pyridin-3-yl)methyl]-2-methyl-1 [(R)-1-[1-(2,2,2-trifluoroethyl)piperidin-4-yl]ethyl]indole-3-carboxamide (CPI-1205), (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide (GSK28 1 6126), (R)-5,8-dichloro-7-(methoxy(oxetan-3-yl)methyl)-2-((4-methoxy-6-methyl-2-oxo-1,2-dihydropyridin-3-yl)methyl)-3,4-dihydroisoquinolin-1(2H)-one (PF-06821497), SHR2554, and combinations thereof.

In further embodiments, the additional therapeutic agent may be, e.g., hydroxyurea. In certain embodiments, the additional therapeutic agent may be selected from the group consisting of, for example, 2-hydroxy-6-((2-(1-isopropyl-1H-pyrazol-5-yl)pyridin-3-yl)methoxy)benzaldehyde (voxelotor, GBT-440), P-Selectin antibodies, L-Glutamine, and combinations thereof.

In some embodiments, the additional therapeutic agent may be, for example, an anti-adhesion agent. For example, in certain embodiments the additional therapeutic agent may be selected from the group consisting of crizanlizumab (SEG101), (2S)-2-[(2R,3R,4S,5S,6R)-3-benzoyloxy-2-[(1R,2R,3S,5R)-3-[(2,4-dioxo-1H-pyrimidine-6-carbonyl)amino]-5-[2-[[2-[2-[2-oxo-2-[(3,6,8-trisulfonaphthalen-1-yl)amino]ethoxy]ethoxy]acetyl]amino]ethylcarbamoyl]-2-[(2S,3S,4R,5S,6S)-3,4,5-trihydroxy-6-methyloxan-2-yl]oxycyclohexyl]oxy-5-hydroxy-6-(hydroxymethyl)oxan-4-yl]oxy-3-cyclohexylpropanoic acid (rivipansel, GMI-1070), sevuparin, 6-[(3I,4S)-4-methyl-1-(pyrimidin-2-ylmethyl)pyrrolidin-3-yl]-1-(oxan-4-yl)-5H-pyrazolo[3,4-d]pyrimidin-4-one (PF-04447943), inclacumab (LC1004-002), 3-[3-[4-(1-aminocyclobutyl)phenyl]-5-phenylimidazo[4,5-b]pyridin-2-yl]pyridin-2-amine (miransertib, ARQ 092), and combinations thereof.

In other embodiments, the additional therapeutic agent may be, for example, an anti-sickling agent. For example, in certain embodiments the additional therapeutic agent may be selected from the group consisting of 2-hydroxy-6-((2-(1-isopropyl-1H-pyrazol-5-yl)pyridin-3-yl)methoxy)benzaldehyde (voxelotor, GBT-440), 6-[(3S,4S)-4-methyl-1-(2-pyrimidinylmethyl)-3-pyrrolidinyl]-3-(tetrahydro-2H-pyran-4-yl)imidazo[1,5-a]pyrazin-8(7H)-one (IMR-687), and combinations thereof.

In further embodiments, the additional therapeutic agent may be, for example, a detoxification agent. For example, in certain embodiments the additional therapeutic agent may be LJPC-401. In some embodiments, the additional therapeutic agent may be selected from, for example, anti-inflammatory agents, anti-thrombotic agents, and combinations thereof. For example, in certain embodiments the additional therapeutic agent may be selected from the group consisting of (1S,2S,3R,5S)-3-[7-{[(1R,2S)-2-(3,4-difluorophenyl)cyclopropyl]amino}-5(propylthio)-3H-[1,2,3]-triazolo[4,5-d]pyrimidin-3-yl]-5-(2-hydroxyethoxy)cyclopentane-1,2-diol (brilinta, tricagrelor), (2R)-3,3,3-trifluoro-2-[[[5-fluoro-2-[1-[(2-fluorophenyl)methyl]-5-(1,2-oxazol-3-yl)pyrazol-3-yl]pyrimidin-4-yl]amino]methyl]-2-hydroxypropanamide (olinciguat), NKTT120, and combinations thereof.

In some embodiments, the additional therapeutic agent may be, for example, sanguinate. In other embodiments, the additional therapeutic agent may be, for example, an agent that causes disruption of PRC2. In further embodiments, the additional therapeutic agent is, for example, AZD9291.

### EXAMPLES

The compounds described herein can be prepared in a number of ways based on the teachings contained herein and synthetic procedures known in the art. The following non-limiting examples illustrate the disclosure.

X-ray powder diffraction was performed using a Bruker D8 Advance equipped with Lynxeye detector in reflection mode (Bragg-Brentano geometry). Samples were prepared on Si zero-return wafers. The parameters for XRPD methods used are listed in Table 1 below.

TABLE 1

| Parameter | Regular Scan | High Resolution Scan |
|---|---|---|
| X-ray wavelength | Cu Kα1, 1.540598 Å | Cu Kα1, 1.540598 Å |
| X-ray tube setting | 40 kV, 40 mA | 40 kV, 40 mA |
| Slit condition | 0.6 mm div. + 2.5° soller | 0.6 mm div. + 2.5° soller |
| Scan mode | Step | Step |
| Scan range (°2θ) | 4-30 | 4-40 |
| Step size (°2θ) | 0.03 | 0.02 |
| Dwell time (s/step) | 0.23 | 0.9 |
| Spin | Yes (0.5 Hz) | Yes (0.5 Hz) |

For some crystalline forms obtained during screening, an XRPD peak at 19.2 degrees 2θ was observed due to a small impurity present in the input material. For clarity, and where applicable, this peak was omitted from Tables 3-31 but can be observed in some Figures.

Differential scanning calorimetry (DSC) and thermogravimetric analysis were performed using a Mettler Toledo DSC³⁺. Samples (3-5 mg) were weighed directly in a hermetic aluminum pan with pinhole and analyzed according to the parameters in Table 2 below.

TABLE 2
Parameters

| Method | Ramp | |
|---|---|---|
| Sample size | 3-5 | mg |
| Heating rate | 10.0° | C./min |
| Temperature range | 30 to 300° | C. |

| | |
|---|---|
| Heating gas | N₂ at 60.00 mL/min |

Dynamic vapor sorption (DVS) analysis was performed using a DVS Intrinsic 1. The sample (25 mg) was loaded into a sample pan, suspended from a microbalance and exposed to a humidified stream of nitrogen gas. The sample was held for a minimum of 5 min at each level and only progressed to the next humidity level if there was <0.002% change in weight between measurements (interval: 60 seconds) or 240 min had elapsed. The following program was used as shown in Table 2B:

TABLE 2B

| | |
|---|---|
| 1) | Equilibration at 50% RH |
| 2) | 50% to 2% (50%, 40%, 30%, 20%, 10% and 2%) |
| 3) | 2% to 95% (2%, 10%, 20%, 30%, 40%, 50%, 60%, 70%, 80%, 90%, |
| | 95%) |
| 4) | 95% to 2% (95%, 80%, 70%, 60%, 50%, 40%, 30%, 20%, 10%, 2%) |
| 5) | 2% to 50% (2%, 10%, 20%, 30%, 40%, 50%) |

Optical microscopy was performed using a Zeiss AxioScope A1 equipped with 2.5×, 10×, 20× and 40× objectives and polarizer. Images are captured through a built-in Axiocam 105 digital camera and processed using ZEN 2 (blue edition) software provided by Zeiss.

### Example 1

Crystalline, Form A material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Approximately 4 g of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, was weighed into a 100 mL vessel followed by 80 mL (20 vol.) of distilled water. The slurry was stirred for about 15 min prior to the addition of 25% ammonia solution, performed dropwise in three aliquots of 5 mL each. The slurry was stirred for 6.5 h, filtered through a cloth filter (˜1.5 min), and washed with 60 mL of water. XRPD analysis of the wet cake indicated that the material was crystalline with a pattern consistent with Form A.

Crystalline Form A material was also prepared as follows. About 30 mg of Form B material was added to a 2 mL vial followed by 0.5 mL of cyclohexane. After stirring at room temperature for two days, the vial was centrifuged and the supernatant decanted. XRPD analysis of the solid (wet and dry) indicated that the material was crystalline with a pattern consistent with Form A.

The XRPD pattern of Form A of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 1. Characteristic peaks include one or more of the peaks shown in Table 3.

TABLE 3

| | | Relative |
|---|---|---|
| 2θ | d-spacing | intensity |
| (deg) | (Å) | (a.u) |
| 5.67 | 15.58 | 25 |
| 8.10 | 10.91 | 15 |
| 8.63 | 10.23 | 6 |
| 8.96 | 9.86 | 100 |
| 12.02 | 7.36 | 52 |
| 12.92 | 6.85 | 6 |
| 14.43 | 6.13 | 12 |
| 16.52 | 5.36 | 17 |
| 16.99 | 5.21 | 5 |
| 17.93 | 4.94 | 11 |
| 20.06 | 4.42 | 13 |
| 20.43 | 4.34 | 7 |
| 21.62 | 4.11 | 9 |
| 22.68 | 3.92 | 6 |
| 23.40 | 3.80 | 6 |
| 24.44 | 3.64 | 7 |

FIG. 2 depicts the differential scanning calorimetry (DSC) profile of crystalline Form A obtained from the preparation in cyclohexane. As shown in FIG. 2, crystalline Form A shows a characteristic endotherm with an onset of about 167° C. and a peak of about 177° C.

Crystalline Form A of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine displayed a thermogravimetric analysis (TGA) profile showing a mass loss of 3.64 wt. % up to 160° C. and an additional loss of 1.97 wt. % from 160 to 250° C. (5.61 wt. % total). Crystalline Form A displayed a dynamic vapor sorption (DVS) profile showing a mass change of about 6.34 wt. % (about 1.48 eq. water) between about 2 to about 92% relative humidity (RH) at 25° C. Crystalline Form A displayed a mixture of irregular and needle-like morphologies by optical microscopy.

### Example 2

Crystalline, Form B material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. The wet cake Form A material obtained from the aqueous preparation in Example 1 was further dried at 50° C. under vacuum overnight. XRPD analysis indicated that the material was crystalline with a pattern consistent with Form B.

The XRPD pattern of Form B of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 3. Characteristic peaks include one or more of the peaks shown in Table 4.

TABLE 4

| | | Relative |
|---|---|---|
| 2θ | d-spacing | intensity |
| (deg) | (Å) | (a.u) |
| 5.70 | 15.49 | 17 |
| 6.21 | 14.22 | 7 |
| 8.13 | 10.87 | 54 |
| 8.65 | 10.22 | 17 |
| 9.00 | 9.82 | 100 |
| 12.10 | 7.31 | 46 |
| 12.93 | 6.84 | 23 |
| 13.52 | 6.54 | 9 |
| 14.30 | 6.19 | 5 |
| 14.51 | 6.10 | 7 |
| 16.52 | 5.36 | 12 |
| 17.23 | 5.14 | 8 |
| 17.99 | 4.93 | 5 |
| 19.70 | 4.50 | 9 |
| 20.17 | 4.40 | 11 |
| 20.52 | 4.32 | 26 |
| 21.67 | 4.10 | 12 |
| 22.71 | 3.91 | 22 |
| 23.54 | 3.78 | 14 |
| 24.47 | 3.63 | 13 |
| 25.71 | 3.46 | 7 |
| 26.79 | 3.32 | 9 |
| 28.29 | 3.15 | 7 |

FIG. 4 depicts the differential scanning calorimetry (DSC) profile of crystalline Form B. As shown in FIG. 4, crystalline Form B shows a characteristic endotherm with an onset of about 68° C. and a peak of about 98° C., a characteristic endotherm with an onset of about 164° C. and a peak of about 168° C., and a characteristic endotherm with an onset of about 168° C. and a peak of about 171° C.

### Example 3

Crystalline, Form C material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. 2.07 g of Form B material was weighed into a 100 mL vessel and 50 mL of EtOH (25 vol.) was added. The slurry was stirred at 450 rpm. After about an hour the slurry had thickened, requiring the further addition of 25 mL of EtOH (12.5 vol). The slurry was stirred for 6 h, filtered, washed with an additional 25 mL, and dried under vacuum (−27.5 in Hg, diaphragm pump) overnight at room temperature. XRPD analysis of the solid indicated that the material was crystalline with a pattern consistent with Form C.

The XRPD pattern of Form C of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 5. Characteristic peaks include one or more of the peaks shown in Table 5.

TABLE 5

| | | Relative |
|---|---|---|
| 2θ | d-spacing | intensity |
| (deg) | (Å) | (a.u) |
| 6.18 | 14.30 | 48 |
| 6.47 | 13.64 | 34 |
| 6.72 | 13.14 | 50 |
| 8.40 | 10.52 | 23 |
| 8.81 | 10.03 | 23 |
| 10.72 | 8.24 | 100 |
| 11.41 | 7.75 | 12 |
| 13.47 | 6.57 | 15 |
| 14.20 | 6.23 | 12 |

FIG. 6 depicts the differential scanning calorimetry (DSC) profile of crystalline Form C. As shown in FIG. 6, crystalline Form C shows a characteristic endotherm with an onset of about 92° C. and a peak of about 107° C., a characteristic endotherm with an onset of about 98° C. and a peak of about 119° C., and a characteristic endotherm with an onset of about 161° C. and a peak of about 173° C.

### Example 4

Crystalline, Form D material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Form C material was dried at 50° C. under vacuum (−30 in Hg, oil pump) overnight. XRPD analysis indicated that the material was crystalline with a pattern consistent with Form D.

The XRPD pattern of Form D of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 7. Characteristic peaks include one or more of the peaks shown in Table 6.

TABLE 6

| | | Relative |
|---|---|---|
| 2θ | d-spacing | intensity |
| (deg) | (Å) | (a.u) |
| 6.28 | 14.07 | 70 |
| 6.69 | 13.21 | 100 |
| 8.38 | 10.54 | 49 |
| 8.98 | 9.84 | 39 |
| 10.04 | 8.81 | 14 |
| 11.09 | 7.97 | 99 |
| 11.32 | 7.81 | 26 |
| 11.82 | 7.48 | 23 |
| 13.38 | 6.61 | 10 |
| 14.11 | 6.27 | 12 |
| 14.42 | 6.14 | 19 |
| 14.83 | 5.97 | 10 |
| 18.00 | 4.93 | 8 |
| 20.10 | 4.41 | 6 |
| 20.57 | 4.31 | 5 |

### Example 5

Crystalline, Form E material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. About 30 mg of Form B material was added to a 2 mL vial followed by 0.5 mL of tetrahydrofuran. After stirring at 50° C. for two days, the vial was centrifuged and the supernatant decanted. XRPD analysis of the solid (wet and dry) indicated that the material was crystalline with a pattern consistent with Form E.

The XRPD pattern of Form E of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 8. Characteristic peaks include one or more of the peaks shown in Table 7.

TABLE 7

| | | Relative |
|---|---|---|
| 2θ | d-spacing | intensity |
| (deg) | (Å) | (a.u) |
| 6.42 | 13.76 | 36 |
| 8.27 | 10.69 | 22 |
| 9.61 | 9.20 | 52 |
| 9.79 | 9.02 | 100 |
| 10.63 | 8.32 | 17 |
| 12.81 | 6.91 | 31 |
| 13.32 | 6.64 | 74 |
| 16.53 | 5.36 | 6 |
| 17.24 | 5.14 | 16 |
| 19.23 | 4.61 | 5 |
| 20.04 | 4.43 | 7 |
| 20.35 | 4.36 | 5 |
| 21.30 | 4.17 | 12 |
| 22.16 | 4.01 | 12 |
| 23.51 | 3.78 | 5 |
| 25.97 | 3.43 | 8 |

FIG. 9 depicts the differential scanning calorimetry (DSC) profile of crystalline Form E. As shown in FIG. 9, crystalline Form E shows a characteristic endotherm with an onset of about 272° C. and a peak of about 274° C.

Crystalline Form E of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine displayed a thermogravimetric analysis (TGA) profile showing a mass loss of about 0.86 wt. % up to melting. Crystalline Form E displayed a dynamic vapor sorption (DVS) profile showing a mass change of about 0.23 wt. % between about 2 to about 92% relative humidity (RH). Crystalline Form E displayed a needle-like morphology by optical microscopy.

### Example 7

Crystalline, Form F material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. About 30 mg of Form B material was added to a 2 mL vial followed by 0.5 mL of acetonitrile. After stirring at room temperature for two days, the vial was centrifuged and the supernatant decanted. XRPD analysis of the solid (wet and dry) indicated that the material was crystalline with a pattern consistent with Form F.

The XRPD pattern of Form F of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 10. Characteristic peaks include one or more of the peaks shown in Table 8.

TABLE 8

| | | Relative |
|---|---|---|
| 2θ | d-spacing | intensity |
| (deg) | (Å) | (a.u) |
| 5.32 | 16.61 | 68 |
| 5.65 | 15.63 | 23 |
| 6.07 | 14.54 | 13 |
| 7.03 | 12.57 | 25 |
| 8.08 | 10.93 | 21 |
| 9.28 | 9.52 | 100 |
| 10.58 | 8.36 | 24 |
| 10.99 | 8.05 | 70 |
| 12.13 | 7.29 | 11 |
| 12.48 | 7.09 | 29 |
| 13.32 | 6.64 | 8 |
| 14.57 | 6.08 | 19 |
| 15.04 | 5.89 | 11 |
| 15.25 | 5.81 | 9 |
| 15.86 | 5.58 | 7 |
| 16.19 | 5.47 | 10 |
| 17.32 | 5.12 | 11 |
| 20.07 | 4.42 | 11 |
| 20.65 | 4.30 | 8 |
| 21.51 | 4.13 | 9 |

### Example 7

Crystalline, Form G material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. About 30 mg of Form B material was added to a 2 mL vial followed by 0.5 mL of isopropyl alcohol. After stirring at 50° C. for two days, the vial was centrifuged and the supernatant decanted. XRPD analysis of the solid (wet and dry) indicated that the material was crystalline with a pattern consistent with Form G.

The XRPD pattern of Form G of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 11. Characteristic peaks include one or more of the peaks shown in Table 9.

TABLE 9

| | | Relative |
|---|---|---|
| 2θ | d-spacing | intensity |
| (deg) | (Å) | (a.u) |
| 4.18 | 21.13 | 16 |
| 4.68 | 18.88 | 9 |
| 6.70 | 13.17 | 23 |
| 7.56 | 11.69 | 64 |
| 8.38 | 10.55 | 100 |
| 8.93 | 9.90 | 82 |
| 9.36 | 9.44 | 14 |
| 10.50 | 8.42 | 20 |
| 11.14 | 7.94 | 14 |
| 11.29 | 7.83 | 11 |
| 11.86 | 7.46 | 7 |
| 12.60 | 7.02 | 31 |
| 12.93 | 6.84 | 19 |
| 13.27 | 6.67 | 47 |
| 15.11 | 5.86 | 18 |
| 16.36 | 5.42 | 5 |
| 16.99 | 5.22 | 14 |
| 17.29 | 5.12 | 9 |
| 18.57 | 4.78 | 12 |
| 18.93 | 4.68 | 8 |
| 20.44 | 4.34 | 8 |
| 20.69 | 4.29 | 11 |
| 21.03 | 4.22 | 12 |
| 21.42 | 4.15 | 19 |
| 21.68 | 4.10 | 16 |

FIG. 12 depicts the differential scanning calorimetry (DSC) profile of crystalline Form G. As shown in FIG. 12, crystalline Form G shows a characteristic endotherm with an onset of about 166° C. and a peak of about 175° C., a characteristic endotherm with an onset of about 241° C. and a peak of about 245° C., a characteristic exotherm with an onset of about 249° C. and a peak of about 251° C., and a characteristic endotherm with an onset of about 267° C. and a peak of about 271° C.

Crystalline Form G of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine displayed a thermogravimetric analysis (TGA) profile showing a mass loss of about 5.99 wt. % up to about 205° C. Crystalline Form G displayed by a mixture of irregular and needle-like morphologies by optical microscopy.

### Example 8

Crystalline, Form H material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. About 30 mg of Form B material was added to a 2 mL vial followed by 0.5 mL of acetone. After stirring at room temperature for two days, the vial was centrifuged and the supernatant decanted. XRPD analysis of the wet solid indicated that the material was crystalline with a pattern consistent with Form H. Drying of the wet material resulted in the conversion of Form H to Form E.

The XRPD pattern of Form H of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 13. Characteristic peaks include one or more of the peaks shown in Table 10.

TABLE 10

| | | Relative |
|---|---|---|
| 2θ | d-spacing | intensity |
| (deg) | (Å) | (a.u) |
| 4.06 | 21.77 | 19 |
| 6.22 | 14.19 | 31 |
| 8.07 | 10.94 | 30 |
| 8.26 | 10.70 | 6 |
| 8.99 | 9.82 | 5 |
| 9.35 | 9.45 | 100 |
| 9.51 | 9.29 | 78 |
| 10.25 | 8.62 | 12 |
| 12.46 | 7.10 | 14 |
| 12.99 | 6.81 | 8 |
| 13.28 | 6.66 | 6 |
| 14.50 | 6.10 | 7 |
| 14.72 | 6.01 | 14 |
| 15.35 | 5.77 | 5 |
| 16.24 | 5.46 | 9 |
| 16.82 | 5.27 | 13 |
| 18.67 | 4.75 | 8 |
| 19.01 | 4.67 | 9 |
| 21.52 | 4.13 | 6 |
| 22.45 | 3.96 | 9 |
| 24.27 | 3.66 | 8 |
| 24.92 | 3.57 | 5 |
| 24.96 | 3.56 | 5 |

### Example 9

Crystalline, Form I material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. About 30 mg of Form B material was added to a 2 mL vial followed by 0.5 mL of water. After stirring at room temperature for two days, the vial was centrifuged and the supernatant decanted. XRPD analysis of the wet solid indicated that the material was crystalline with a pattern consistent with Form I. Drying of the wet material under resulted in the conversion of Form I to Form M.

The XRPD pattern of Form I of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 14. Characteristic peaks include one or more of the peaks shown in Table 11.

TABLE 11

| | | Relative |
|---|---|---|
| 2θ | d-spacing | intensity |
| (deg) | (Å) | (a.u) |
| 4.46 | 19.78 | 26 |
| 4.85 | 18.19 | 65 |
| 5.19 | 17.03 | 40 |
| 5.51 | 16.04 | 10 |
| 6.10 | 14.48 | 58 |
| 6.68 | 13.22 | 8 |
| 7.47 | 11.82 | 52 |
| 7.81 | 11.32 | 70 |
| 8.13 | 10.87 | 99 |
| 8.84 | 10.00 | 80 |
| 9.11 | 9.70 | 97 |
| 9.57 | 9.24 | 91 |
| 10.34 | 8.55 | 100 |
| 11.10 | 7.96 | 24 |
| 11.36 | 7.79 | 21 |
| 11.73 | 7.54 | 22 |
| 12.13 | 7.29 | 83 |
| 12.57 | 7.04 | 12 |
| 13.19 | 6.71 | 77 |
| 14.12 | 6.27 | 44 |
| 14.44 | 6.13 | 49 |
| 14.70 | 6.02 | 60 |
| 15.17 | 5.83 | 50 |
| 15.46 | 5.73 | 20 |
| 16.22 | 5.46 | 24 |
| 16.82 | 5.27 | 8 |
| 17.32 | 5.11 | 18 |
| 17.70 | 5.01 | 35 |
| 18.21 | 4.87 | 9 |
| 19.25 | 4.61 | 37 |
| 19.89 | 4.46 | 14 |
| 20.15 | 4.40 | 9 |
| 20.63 | 4.30 | 21 |
| 20.90 | 4.25 | 66 |
| 21.27 | 4.17 | 13 |
| 21.77 | 4.08 | 7 |
| 22.25 | 3.99 | 50 |
| 22.51 | 3.95 | 12 |
| 22.93 | 3.88 | 17 |
| 23.52 | 3.78 | 6 |
| 24.18 | 3.68 | 9 |
| 25.54 | 3.48 | 9 |
| 26.01 | 3.42 | 6 |
| 26.58 | 3.35 | 37 |
| 27.60 | 3.23 | 8 |
| 28.73 | 3.10 | 10 |
| 29.55 | 3.02 | 12 |
| 29.81 | 2.99 | 8 |

### Example 10

Crystalline, Form J material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. About 30 mg of Form B material was added to a 2 mL vial followed by 0.5 mL of EtOH. After stirring at 50° C. for two days, the vial was centrifuged and the supernatant decanted. XRPD analysis of the wet solid indicated that the material was crystalline with a pattern consistent with Form J. Drying of the wet material resulted in the conversion of Form J to Form D.

The XRPD pattern of Form J of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 15. Characteristic peaks include one or more of the peaks shown in Table 12.

TABLE 12

| | | Relative |
|---|---|---|
| 2θ | d-spacing | intensity |
| (deg) | (Å) | (a.u) |
| 5.21 | 16.95 | 11 |
| 6.33 | 13.94 | 34 |
| 6.49 | 13.60 | 13 |
| 7.82 | 11.29 | 10 |
| 8.12 | 10.88 | 9 |
| 8.29 | 10.66 | 12 |
| 8.54 | 10.35 | 15 |
| 10.64 | 8.31 | 100 |
| 11.21 | 7.89 | 14 |
| 14.25 | 6.21 | 7 |
| 14.66 | 6.04 | 7 |
| 17.41 | 5.09 | 5 |
| 21.12 | 4.20 | 5 |
| 21.19 | 4.19 | 5 |

### Example 11

Crystalline, Form K material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. About 30 mg of Form B material was added to a 2 mL vial followed by 0.5 mL of water. After stirring at 50° C. for two days, the vial was centrifuged and the supernatant decanted. XRPD analysis of the wet solid indicated that the material was crystalline with a pattern consistent with Form K. Drying of the wet material resulted in the conversion of Form K to Form N.

The XRPD pattern of Form K of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 16. Characteristic peaks include one or more of the peaks shown in Table 13.

TABLE 13

| | | Relative |
|---|---|---|
| 2θ | d-spacing | intensity |
| (deg) | (Å) | (a.u) |
| 5.46 | 16.17 | 32 |
| 6.37 | 13.87 | 13 |
| 7.06 | 12.50 | 42 |
| 8.75 | 10.09 | 100 |
| 9.28 | 9.52 | 25 |
| 10.63 | 8.32 | 83 |
| 10.82 | 8.17 | 45 |
| 11.12 | 7.95 | 12 |
| 11.55 | 7.66 | 49 |
| 12.06 | 7.33 | 14 |
| 12.64 | 7.00 | 14 |
| 13.31 | 6.65 | 21 |
| 14.05 | 6.30 | 28 |
| 14.86 | 5.96 | 37 |
| 16.01 | 5.53 | 10 |
| 16.21 | 5.46 | 24 |
| 16.50 | 5.37 | 6 |
| 16.87 | 5.25 | 11 |
| 17.04 | 5.20 | 9 |
| 17.44 | 5.08 | 9 |
| 17.79 | 4.98 | 13 |
| 18.40 | 4.82 | 6 |
| 18.98 | 4.67 | 4 |
| 19.64 | 4.52 | 15 |
| 20.86 | 4.26 | 6 |
| 21.10 | 4.21 | 18 |
| 21.41 | 4.15 | 6 |
| 21.92 | 4.05 | 17 |
| 22.26 | 3.99 | 5 |
| 22.69 | 3.92 | 5 |
| 23.10 | 3.85 | 6 |
| 25.52 | 3.49 | 7 |
| 26.08 | 3.41 | 4 |
| 26.56 | 3.35 | 8 |
| 27.39 | 3.25 | 5 |
| 28.48 | 3.13 | 8 |

### Example 12

Crystalline, Form L material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. About 30 mg of Form B material was added to a 2 mL vial followed by 0.5 mL of acetonitrile. After stirring at room temperature for three days, the solids were allowed to settle and the supernatant was recovered. The supernatant was evaporated to dryness at 50° C. in atmosphere overnight and further dried at 50° C. under vacuum for 4 h. XRPD analysis of the solid indicated that the material was crystalline with a pattern consistent with Form L.

The XRPD pattern of Form L of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 17. Characteristic peaks include one or more of the peaks shown in Table 14.

TABLE 14

| | | Relative |
|---|---|---|
| 2θ | d-spacing | intensity |
| (deg) | (Å) | (a.u) |
| 5.44 | 16.24 | 12 |
| 6.96 | 12.69 | 20 |
| 7.51 | 11.76 | 64 |
| 8.93 | 9.90 | 100 |
| 9.13 | 9.68 | 14 |
| 9.64 | 9.17 | 24 |
| 10.00 | 8.83 | 72 |
| 11.11 | 7.96 | 14 |
| 12.51 | 7.07 | 16 |
| 13.91 | 6.36 | 5 |
| 14.72 | 6.01 | 5 |
| 15.02 | 5.89 | 37 |
| 18.21 | 4.87 | 12 |

### Example 13

Crystalline, Form M material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Form I material (wet solid) obtained in Example 9 was dried at 50° C. under vacuum. XRPD analysis of the dry solid indicated that the material was crystalline with a pattern consistent with Form M.

The XRPD pattern of Form M of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 18. Characteristic peaks include one or more of the peaks shown in Table 15.

TABLE 15

| | | Relative |
|---|---|---|
| 2θ | d-spacing | intensity |
| (deg) | (Å) | (a.u) |
| 4.84 | 18.26 | 68 |
| 5.56 | 15.90 | 21 |
| 7.29 | 12.12 | 100 |
| 9.08 | 9.73 | 55 |
| 9.61 | 9.20 | 67 |
| 11.09 | 7.97 | 54 |
| 12.39 | 7.14 | 82 |
| 13.16 | 6.72 | 6 |
| 13.84 | 6.39 | 22 |
| 14.53 | 6.09 | 20 |
| 16.38 | 5.41 | 15 |
| 16.68 | 5.31 | 14 |
| 17.14 | 5.17 | 5 |
| 18.03 | 4.92 | 5 |
| 19.38 | 4.58 | 14 |
| 21.80 | 4.07 | 5 |

### Example 14

Crystalline, Form N material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Form K material (wet solid) obtained in Example 11 was dried at 50° C. under vacuum. XRPD analysis of the dry solid indicated that the material was crystalline with a pattern consistent with Form N.

The XRPD pattern of Form N of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 19. Characteristic peaks include one or more of the peaks shown in Table 16.

TABLE 16

| | | Relative |
|---|---|---|
| 2θ | d-spacing | intensity |
| (deg) | (Å) | (a.u) |
| 6.53 | 13.53 | 45 |
| 7.23 | 12.22 | 22 |
| 8.91 | 9.92 | 100 |
| 9.31 | 9.49 | 19 |
| 9.83 | 8.99 | 13 |
| 11.02 | 8.02 | 44 |
| 11.96 | 7.40 | 28 |
| 13.71 | 6.46 | 40 |
| 14.42 | 6.14 | 12 |
| 15.01 | 5.90 | 8 |
| 16.29 | 5.44 | 8 |
| 16.88 | 5.25 | 5 |
| 17.21 | 5.15 | 5 |
| 20.23 | 4.39 | 9 |
| 20.93 | 4.24 | 9 |
| 22.85 | 3.89 | 5 |
| 23.56 | 3.77 | 5 |

FIG. 20 depicts the differential scanning calorimetry (DSC) profile of crystalline Form N. As shown in FIG. 20, crystalline Form N shows a characteristic endotherm with an onset of about 39° C. and a peak of about 58° C., a characteristic endotherm with an onset of about 78° C. and a peak of about 94° C., a characteristic endotherm with an onset of about 171° C. and a peak of about 186° C., a characteristic endotherm with an onset of about 245° C. and a peak of about 249° C., and a characteristic endotherm with an onset of about 268° C. and a peak of about 271° C.

### Example 15

Crystalline, Form O material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. About 30 mg of Form B material was added to a 2 mL vial followed by 0.5 mL of dioxane. After stirring at room temperature for two days, the vial was centrifuged and the supernatant decanted. XRPD analysis of the wet solid indicated that the material was crystalline with a pattern consistent with Form O. Drying of the wet material resulted in the conversion of Form O to Form P.

The XRPD pattern of Form O of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 21. Characteristic peaks include one or more of the peaks shown in Table 17.

TABLE 17

| | | Relative |
|---|---|---|
| 2θ | d-spacing | intensity |
| (deg) | (Å) | (a.u) |
| 5.62 | 15.72 | 24 |
| 7.91 | 11.17 | 5 |
| 8.84 | 10.00 | 100 |
| 11.85 | 7.46 | 42 |
| 14.24 | 6.22 | 25 |
| 16.28 | 5.44 | 35 |
| 16.76 | 5.29 | 16 |
| 17.67 | 5.02 | 23 |
| 19.77 | 4.49 | 15 |
| 20.15 | 4.40 | 5 |
| 21.31 | 4.17 | 13 |
| 23.08 | 3.85 | 7 |
| 24.11 | 3.69 | 5 |

### Example 16

Crystalline, Form P material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Form O material (wet solid) obtained in Example 15 was dried at 50° C. under vacuum. XRPD analysis of the dry solid indicated that the material was crystalline with a pattern consistent with Form P.

The XRPD pattern of Form P of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 22. Characteristic peaks include one or more of the peaks shown in Table 18.

TABLE 18

| | | Relative |
|---|---|---|
| 2θ | d-spacing | intensity |
| (deg) | (Å) | (a.u) |
| 5.65 | 15.62 | 22 |
| 7.98 | 11.07 | 7 |
| 8.91 | 9.91 | 100 |
| 11.95 | 7.40 | 43 |
| 14.36 | 6.16 | 24 |
| 16.43 | 5.39 | 35 |
| 16.91 | 5.24 | 9 |
| 17.82 | 4.97 | 20 |
| 19.95 | 4.45 | 15 |
| 20.32 | 4.37 | 5 |
| 21.49 | 4.13 | 15 |
| 23.29 | 3.82 | 8 |
| 24.30 | 3.66 | 5 |
| 25.28 | 3.52 | 5 |

FIG. 23 depicts the differential scanning calorimetry (DSC) profile of crystalline Form P. As shown in FIG. 18, crystalline Form P shows a characteristic endotherm with an onset of about 47° C. and a peak of about 85° C., and a characteristic endotherm with an onset of about 179° C. and a peak of about 189° C.

### Example 17

Crystalline, Form Q material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Form C material (30 mg) was dissolved in 1:1 dimethyl sulfoxide:isopropyl alcohol (5 volumes) at 55° C. The stirred solution was cooled from 55° C. to room temperature at 5° C. per hour, accomplished by reducing the hot plate temperature by 2.5° C. every 30 minutes. The slurry solids were then filtered. XRPD analysis of the solid (wet and dry) indicated that the material was crystalline with a pattern consistent with Form Q.

The XRPD pattern of Form Q of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 24. Characteristic peaks include one or more of the peaks shown in Table 19.

TABLE 19

| | | Relative |
|---|---|---|
| 2θ | d-spacing | intensity |
| (deg) | (Å) | (a.u) |
| 7.49 | 11.80 | 28 |
| 8.50 | 10.39 | 100 |
| 9.80 | 9.02 | 9 |
| 12.98 | 6.82 | 8 |
| 14.76 | 6.00 | 11 |
| 14.97 | 5.91 | 18 |
| 15.79 | 5.61 | 12 |
| 17.71 | 5.00 | 10 |
| 18.63 | 4.76 | 10 |
| 20.49 | 4.33 | 5 |
| 22.16 | 4.01 | 5 |

FIG. 25 depicts the differential scanning calorimetry (DSC) profile of crystalline Form Q. As shown in FIG. 25, crystalline Form Q shows a characteristic endotherm with an onset of about 145° C. and a peak of about 152° C., a characteristic endotherm with an onset of about 244° C. and a peak of about 249, and a characteristic endotherm with an onset of about 266° C. and a peak of about 271° C.

### Example 18

Crystalline, Form R material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Form C material (30 mg) was dissolved in methanol (110 volumes) at 55° C. The stirred solution was cooled from 50° C. to room temperature at 5° C. per hour, accomplished by reducing the hot plate temperature by 2.5° C. every 30 minutes. The slurry solids were then filtered. XRPD analysis of the wet solid indicated that the material was crystalline with a pattern consistent with Form R. Drying of the wet material under resulted in the conversion of Form R to Form S.

The XRPD pattern of Form R of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 26. Characteristic peaks include one or more of the peaks shown in Table 20.

TABLE 20

| | | Relative |
|---|---|---|
| 2θ | d-spacing | intensity |
| (deg) | (Å) | (a.u) |
| 6.16 | 14.34 | 31 |
| 8.03 | 11.00 | 41 |
| 9.09 | 9.72 | 75 |
| 9.62 | 9.19 | 78 |
| 10.25 | 8.63 | 100 |
| 10.86 | 8.14 | 36 |
| 11.52 | 7.67 | 57 |
| 12.29 | 7.20 | 26 |
| 12.89 | 6.86 | 47 |
| 13.76 | 6.43 | 7 |
| 14.05 | 6.30 | 25 |
| 14.40 | 6.15 | 8 |
| 15.22 | 5.82 | 6 |
| 15.72 | 5.63 | 34 |
| 16.96 | 5.23 | 17 |
| 18.69 | 4.74 | 9 |
| 19.30 | 4.60 | 19 |
| 20.64 | 4.30 | 12 |
| 21.21 | 4.19 | 38 |
| 21.72 | 4.09 | 6 |
| 23.83 | 3.73 | 8 |
| 25.19 | 3.53 | 6 |

### Example 19

Crystalline, Form S material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Form R material (wet solid) obtained in Example 18 was dried at 50° C. under vacuum. XRPD analysis of the dry solid indicated that the material was crystalline with a pattern consistent with Form S.

The XRPD pattern of Form S of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 27. Characteristic peaks include one or more of the peaks shown in Table 21.

TABLE 21

| | | Relative |
|---|---|---|
| 2θ | d-spacing | intensity |
| (deg) | (Å) | (a.u) |
| 5.58 | 15.83 | 14 |
| 6.17 | 14.30 | 9 |
| 7.29 | 12.11 | 13 |
| 8.04 | 10.99 | 22 |
| 8.34 | 10.59 | 83 |
| 9.43 | 9.37 | 84 |
| 9.76 | 9.06 | 100 |
| 10.67 | 8.29 | 77 |
| 11.14 | 7.94 | 24 |
| 11.33 | 7.80 | 14 |
| 11.89 | 7.44 | 55 |
| 13.35 | 6.63 | 48 |
| 14.13 | 6.26 | 9 |
| 14.59 | 6.07 | 19 |
| 14.87 | 5.95 | 25 |
| 15.62 | 5.67 | 26 |
| 16.79 | 5.28 | 26 |
| 17.84 | 4.97 | 7 |
| 19.34 | 4.59 | 11 |
| 19.76 | 4.49 | 26 |
| 20.07 | 4.42 | 31 |
| 20.63 | 4.30 | 39 |
| 21.77 | 4.08 | 41 |
| 22.18 | 4.01 | 27 |
| 23.07 | 3.85 | 13 |
| 23.64 | 3.76 | 24 |
| 24.36 | 3.65 | 36 |
| 24.85 | 3.58 | 23 |
| 26.66 | 3.34 | 11 |
| 27.69 | 3.22 | 7 |
| 27.94 | 3.19 | 6 |

### Example 20

Crystalline, Form T material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Form C material (30 mg) was dissolved in 9:1 isopropyl alcohol:water (42 volumes) at 55° C. The stirred solution was cooled from 55° C. to room temperature at 5° C. per hour, accomplished by reducing the hot plate temperature by 2.5° C. every 30 minutes. The slurry solids were then filtered. XRPD analysis of the wet solid indicated that the material was crystalline with a pattern consistent with Form T. Drying of the wet material resulted in the conversion of Form T to Form D.

The XRPD pattern of Form T of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 28. Characteristic peaks include one or more of the peaks shown in Table 22.

TABLE 22

| | | Relative |
|---|---|---|
| 2θ | d-spacing | intensity |
| (deg) | (Å) | (a.u) |
| 5.26 | 16.78 | 10 |
| 6.48 | 13.64 | 100 |
| 8.00 | 11.04 | 37 |
| 8.50 | 10.39 | 35 |
| 10.74 | 8.23 | 67 |
| 11.29 | 7.83 | 8 |
| 12.99 | 6.81 | 6 |
| 13.47 | 6.57 | 12 |
| 14.28 | 6.20 | 16 |
| 14.67 | 6.03 | 9 |
| 17.40 | 5.09 | 7 |
| 20.52 | 4.32 | 5 |
| 21.01 | 4.22 | 8 |

### Example 21

Crystalline, Form U material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. Tetrahydrofuran was added to 30 mg of Form C material at room temperature until complete dissolution. Twice the volume of heptane antisolvent was then added to the stirred tetrahydrofuran solution dropwise in 4 portions over 60 minutes. The resulting slurry solids were collected by filtration. XRPD analysis of the solid (wet and dry) indicated that the material was crystalline with a pattern consistent with Form U.

The XRPD pattern of Form U of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 29. Characteristic peaks include one or more of the peaks shown in Table 23.

TABLE 23

| | | Relative |
|---|---|---|
| 2θ | d-spacing | intensity |
| (deg) | (Å) | (a.u) |
| 8.11 | 10.90 | 16 |
| 9.46 | 9.34 | 44 |
| 9.76 | 9.06 | 100 |
| 12.68 | 6.97 | 54 |
| 14.80 | 5.98 | 0 |
| 16.22 | 5.46 | 10 |
| 16.94 | 5.23 | 19 |
| 18.94 | 4.68 | 8 |
| 19.52 | 4.54 | 12 |
| 20.11 | 4.41 | 9 |
| 21.21 | 4.19 | 15 |
| 22.00 | 4.04 | 12 |
| 22.94 | 3.87 | 8 |
| 23.66 | 3.76 | 5 |
| 25.47 | 3.49 | 9 |
| 26.11 | 3.41 | 5 |

FIG. 30 depicts the differential scanning calorimetry (DSC) profile of crystalline Form U. As shown in FIG. 30, crystalline Form U shows a characteristic endotherm with an onset of about 246° C. and a peak of about 250° C., a characteristic exotherm with an onset of about 253° C. and a peak of about 255° C., and a characteristic endotherm with an onset of about 270° C. and a peak of about 274° C.

Crystalline Form U of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine may be characterized by a thermogravimetric analysis (TGA) profile showing a mass loss of about 0.007 wt. % up to about 255° C. Crystalline Form U displayed a dynamic vapor sorption (DVS) profile showing a mass change of about 0.38 wt. % between about 2 to about 92% relative humidity (RH) at 25° C. Crystalline Form U displayed an irregular morphology by optical microscopy.

### Example 22

Crystalline, Form V material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. N,N-dimethylacetamide was added to 30 mg of Form C material at room temperature until complete dissolution. Twice the volume of water antisolvent was then added to the stirred tetrahydrofuran solution dropwise in 4 portions over 60 minutes. The resulting slurry solids were collected by filtration. XRPD analysis of the solid (wet and dry) indicated that the material was crystalline with a pattern consistent with Form V.

The XRPD pattern of Form V of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 31. Characteristic peaks include one or more of the peaks shown in Table 24.

TABLE 24

| | | Relative |
|---|---|---|
| 2θ | d-spacing | intensity |
| (deg) | (Å) | (a.u) |
| 5.73 | 15.42 | 5 |
| 6.84 | 12.91 | 8 |
| 8.24 | 10.72 | 49 |
| 9.01 | 9.81 | 100 |
| 9.94 | 8.89 | 8 |
| 10.15 | 8.71 | 11 |
| 13.46 | 6.57 | 26 |
| 13.93 | 6.35 | 18 |
| 14.91 | 5.94 | 31 |
| 15.63 | 5.67 | 5 |
| 16.48 | 5.37 | 5 |
| 16.93 | 5.23 | 6 |
| 18.31 | 4.84 | 9 |
| 18.72 | 4.74 | 13 |
| 19.09 | 4.64 | 7 |
| 19.65 | 4.51 | 12 |
| 20.42 | 4.35 | 13 |
| 21.40 | 4.15 | 25 |
| 21.70 | 4.09 | 10 |
| 22.21 | 4.00 | 8 |
| 24.07 | 3.69 | 10 |

FIG. 32 depicts the differential scanning calorimetry (DSC) profile of crystalline Form V. As shown in FIG. 32, crystalline Form V shows a characteristic endotherm with an onset of about 165° C. and a peak of about 176° C., a characteristic exotherm with an onset of about 182° C. and a peak of about 191° C., a characteristic endotherm with an onset of about 198° C. and a peak of about 204° C., a characteristic endotherm with an onset of about 247° C. and a peak of about 251° C., a characteristic exotherm with an onset of about 255° C. and a peak of about 257° C., and a characteristic endotherm with an onset of about 270° C. and a peak of about 274° C.

### Example 23

Crystalline, Form W material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. 9:1 isopropanol:water was added to 30 mg of Form C material at room temperature until complete dissolution. The solution was added all at once to twice the volume of water antisolvent. The resulting slurry solids were collected by filtration. XRPD analysis of the wet solid indicated that the material was crystalline with a pattern consistent with Form K. Drying of the wet material resulted in the conversion of Form K to Form W.

The XRPD pattern of Form W of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 33. Characteristic peaks include one or more of the peaks shown in Table 25.

TABLE 25

| | | Relative |
|---|---|---|
| 2θ | d-spacing | intensity |
| (deg) | (Å) | (a.u) |
| 6.40 | 13.80 | 43 |
| 7.12 | 12.40 | 21 |
| 8.87 | 9.96 | 100 |
| 9.42 | 9.38 | 22 |
| 9.83 | 9.00 | 9 |
| 10.64 | 8.30 | 25 |
| 10.85 | 8.15 | 27 |
| 11.77 | 7.51 | 17 |
| 12.96 | 6.83 | 5 |
| 13.49 | 6.56 | 33 |
| 14.24 | 6.22 | 8 |
| 15.11 | 5.86 | 10 |
| 16.42 | 5.40 | 7 |
| 16.42 | 5.40 | 7 |
| 16.56 | 5.35 | 8 |
| 19.93 | 4.45 | 7 |
| 20.61 | 4.31 | 6 |

### Example 24

Crystalline, Form X material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Approximately 30 mg of Form C material was weighed in a 2 mL vial and heated (dry) to 190° C., resulting in conversion from a flowable white solid to an orange glass. XRPD analysis of the glass indicated that the material was amorphous. The vial was cooled to room temperature and charged with a 5 mm stir bar. Water (500 μL) was then added and the mixture was stirred at room temperature. The mixture was sampled as soon as sufficient slurry solid was observed. The resulting slurry solid was collected by filtration. XRPD analysis of the wet solid indicated that the material was crystalline with a pattern consistent with Form X.

The XRPD pattern of Form X of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 34. Characteristic peaks include one or more of the peaks shown in Table 26.

TABLE 26

| | | Relative |
|---|---|---|
| 2θ | d-spacing | intensity |
| (deg) | (Å) | (a.u) |
| 4.51 | 19.57 | 94 |
| 7.06 | 12.51 | 82 |
| 9.13 | 9.68 | 100 |
| 10.54 | 8.38 | 29 |
| 11.76 | 7.52 | 15 |
| 13.69 | 6.46 | 54 |
| 14.00 | 6.32 | 40 |
| 16.13 | 5.49 | 23 |

### Example 25

Crystalline, Form Y material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Form C material (30 mg) was dissolved in methanol (110 volumes) at 55° C. The solution was transferred to an ice-water bath at 0° C. without mixing. After 10 min in the ice-water bath, mixing was resumed. Once precipitation was observed at 0° C., the resulting slurry was filtered immediately. XRPD analysis of the solid (wet and dry) indicated that the material was crystalline with a pattern consistent with Form Y.

The XRPD pattern of Form Y of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 35. Characteristic peaks include one or more of the peaks shown in Table 27.

TABLE 27

| | | Relative |
|---|---|---|
| 2θ | d-spacing | intensity |
| (deg) | (Å) | (a.u) |
| 6.89 | 12.82 | 47 |
| 8.94 | 9.89 | 84 |
| 10.35 | 8.54 | 100 |
| 14.44 | 6.13 | 13 |
| 20.54 | 4.32 | 24 |
| 20.57 | 4.31 | 26 |

### Example 26

Crystalline, Form Z material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Approximately 30 mg of Form C material was weighed in a 2 mL vial and heated (dry) to 190° C., resulting in conversion from a flowable white solid to an orange glass. XRPD analysis of the glass indicated that the material was amorphous. The vial was cooled to room temperature and charged with a 5 mm stir bar. Acetonitrile (500 μL) was then added and the mixture was stirred at room temperature. The mixture was sampled as soon as sufficient slurry solid was observed. The resulting slurry solid was collected by filtration. XRPD analysis of the wet solid indicated that the material was crystalline with a pattern consistent with Form Z. Drying of the wet solid resulted in the conversion of Form Z to Form α.

The XRPD pattern of Form Z of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 36. Characteristic peaks include one or more of the peaks shown in Table 28.

TABLE 28

| | | Relative |
|---|---|---|
| 2θ | d-spacing | intensity |
| (deg) | (Å) | (a.u) |
| 4.25 | 20.80 | 9 |
| 5.48 | 16.12 | 25 |
| 6.91 | 12.78 | 7 |
| 7.80 | 11.32 | 11 |
| 8.42 | 10.50 | 100 |
| 8.90 | 9.93 | 63 |
| 9.33 | 9.48 | 31 |
| 9.69 | 9.12 | 51 |
| 9.96 | 8.88 | 11 |
| 10.27 | 8.60 | 10 |
| 10.89 | 8.12 | 6 |
| 11.74 | 7.53 | 18 |
| 11.89 | 7.44 | 36 |
| 12.49 | 7.08 | 8 |
| 12.81 | 6.91 | 5 |
| 14.66 | 6.04 | 18 |
| 14.82 | 5.97 | 15 |
| 15.60 | 5.68 | 6 |
| 16.54 | 5.36 | 6 |
| 17.86 | 4.96 | 18 |
| 19.50 | 4.55 | 8 |
| 19.79 | 4.48 | 8 |
| 20.06 | 4.42 | 12 |
| 21.24 | 4.18 | 8 |
| 22.05 | 4.03 | 15 |
| 24.65 | 3.61 | 7 |
| 24.73 | 3.60 | 6 |

### Example 27

Crystalline, Form α material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Form Z material (wet solid) obtained in Example 16 was dried at 50° C. under vacuum. XRPD analysis of the dry solid indicated that the material was crystalline with a pattern consistent with Form α.

The XRPD pattern of Form α of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 37. Characteristic peaks include one or more of the peaks shown in Table 29.

TABLE 29

| | | Relative |
|---|---|---|
| | d-spacing | intensity |
| 20 (deg) | (A) | (a.u) |
| 4.27 | 20.66 | 50 |
| 5.44 | 16.23 | 8 |
| 6.09 | 14.49 | 10 |
| 7.17 | 12.32 | 12 |
| 8.52 | 10.37 | 16 |
| 9.17 | 9.63 | 36 |
| 9.25 | 9.55 | 43 |
| 9.62 | 9.19 | 100 |
| 9.66 | 9.15 | 85 |
| 10.97 | 8.06 | 12 |
| 12.37 | 7.15 | 37 |
| 12.77 | 6.92 | 19 |
| 13.40 | 6.60 | 5 |
| 14.08 | 6.29 | 8 |
| 14.66 | 6.04 | 17 |
| 15.36 | 5.77 | 21 |
| 16.05 | 5.52 | 17 |
| 16.40 | 5.40 | 9 |
| 17.05 | 5.20 | 12 |
| 19.36 | 4.58 | 9 |
| 19.97 | 4.44 | 10 |
| 20.52 | 4.33 | 9 |

### Example 28

Crystalline, Form β material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. A 2 mL vial was charged with 6 mg of Form G material and 2 mL of fasted state intestinal fluid (FaSSIF) at room temperature. A 10 mm stir bar was added and the mixture stirred at 37° C. for 24 h. The slurry solids were collected my filtration. XRPD analysis of the solid indicated that the material was crystalline with a pattern consistent with Form β.

The XRPD pattern of Form β of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 38. Characteristic peaks include one or more of the peaks shown in Table 30.

TABLE 30

| | | Relative |
|---|---|---|
| 2θ | d-spacing | intensity |
| (deg) | (Å) | (a.u) |
| 5.83 | 15.16 | 16 |
| 6.71 | 13.16 | 6 |
| 8.88 | 9.95 | 100 |
| 10.08 | 8.77 | 27 |
| 12.12 | 7.30 | 16 |
| 13.45 | 6.58 | 24 |
| 14.66 | 6.04 | 21 |
| 16.82 | 5.27 | 9 |
| 17.81 | 4.98 | 7 |
| 20.46 | 4.34 | 11 |
| 20.96 | 4.23 | 5 |
| 24.34 | 3.65 | 5 |

### Example 29

Crystalline, Form γ material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Approximately 30 mg of Form C material was weighed into a milling capsule and 1 vol. of ethyl acetate was added along with a ¼″ steel ball as milling media. The solid was milled with a Wig-L-Bug at 3500 RPM for 30 s, then collected. XRPD analysis of the solid indicated that the material was crystalline with a pattern consistent with Form γ.

The XRPD pattern of Form γ of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine is shown in FIG. 39. Characteristic peaks include one or more of the peaks shown in Table 31.

TABLE 31

| | | Relative |
|---|---|---|
| 2θ | d-spacing | intensity |
| (deg) | (Å) | (a.u) |
| 5.15 | 17.16 | 12 |
| 6.33 | 13.95 | 77 |
| 6.64 | 13.30 | 39 |
| 7.76 | 11.39 | 84 |
| 8.38 | 10.54 | 96 |
| 8.90 | 9.93 | 100 |
| 9.29 | 9.51 | 29 |
| 9.64 | 9.17 | 26 |
| 10.37 | 8.52 | 56 |
| 10.62 | 8.33 | 47 |
| 11.17 | 7.92 | 20 |
| 12.65 | 6.99 | 37 |
| 13.29 | 6.66 | 56 |
| 17.04 | 5.20 | 16 |
| 18.58 | 4.77 | 8 |
| 18.77 | 4.72 | 7 |
| 19.01 | 4.66 | 11 |
| 19.87 | 4.46 | 11 |
| 20.32 | 4.37 | 36 |
| 21.07 | 4.21 | 61 |
| 21.72 | 4.09 | 40 |
| 23.51 | 3.78 | 36 |
| 25.93 | 3.43 | 30 |

### Example 30

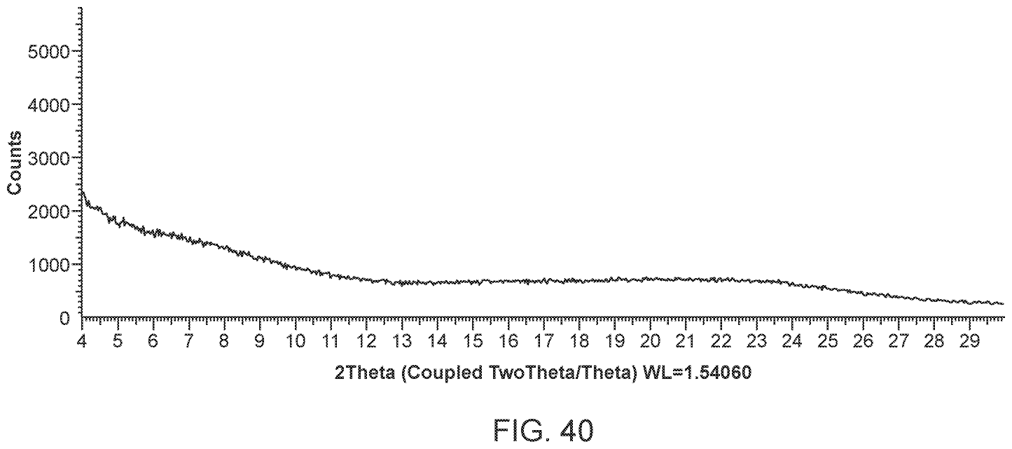

Amorphous form material of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine was prepared as follows. Approximately 30 mg of Form C material was weighed in a 2 mL vial and heated (dry) to 190° C., resulting in conversion from a flowable white solid to an orange glass. XRPD analysis of the glass indicated that the material was amorphous. The XRPD pattern of the amorphous material is shown in FIG. 40.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form E), characterized by a powder X-ray diffraction pattern having a characteristic peak in degrees 2θ at about 9.8.

2. The crystalline form of claim 1, characterized by a powder X-ray diffraction pattern having characteristic peaks in degrees 2θ at about 9.6, 9.8, and 13.3.

3. The crystalline form of claim 2, characterized by a powder X-ray diffraction pattern having characteristic peaks in degrees 2θ at about 6.4, 8.3, 9.6, 9.8, 12.8, and 13.3.

4. The crystalline form of claim 3, characterized by a powder X-ray diffraction pattern having characteristic peaks in degrees 2θ at about 6.4, 8.3, 9.6, 9.8, 10.6, 12.8, 13.3, 17.2, 20.0, 21.3, 22.2, and 26.0.

5. The crystalline form of any one of claims 1-4, wherein the powder X-ray diffraction pattern was obtained using Cu Kα radiation.

6. The crystalline form of any one of claims 1-5, characterized by a differential scanning calorimetry (DSC) profile having a characteristic endotherm with an onset of about 272° C. and a peak of about 274° C.

7. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form A), characterized by a powder X-ray diffraction pattern having characteristic peaks in degrees 2θ at about 5.7, 8.1, 8.6, 9.0, 12.0, 14.4, 16.5, 17.9, 20.1, 20.4, 21.6, and 24.4, wherein the powder X-ray diffraction pattern was obtained using Cu Kα radiation.

8. The crystalline form of claim 7, characterized by a differential scanning calorimetry (DSC) profile having a characteristic endotherm with an onset of about 167° C. and a peak of about 177° C.

9. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form B), characterized by a powder X-ray diffraction pattern having characteristic peaks in degrees 2θ at about 5.7, 8.1, 8.6, 9.0, 12.1, 12.9, 16.5, 20.5, 21.7, 22.7, 23.5, and 24.5, wherein the powder X-ray diffraction pattern was obtained using Cu Kα radiation.

10. The crystalline form of claim 9, characterized by a differential scanning calorimetry (DSC) profile having a characteristic endotherm with an onset of about 68° C. and a peak of about 98° C., a characteristic endotherm with an onset of about 164° C. and a peak of about 168° C., and a characteristic endotherm with an onset of about 168° C. and a peak of about 171° C.

11. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form C), characterized by a powder X-ray diffraction pattern having characteristic peaks in degrees 2θ at about 6.2, 6.5, 6.7, 8.4, 8.8, 10.7, 11.4, 13.5, and 14.2, wherein the powder X-ray diffraction pattern was obtained using Cu Kα radiation.

12. The crystalline form of claim 11, characterized by a differential scanning calorimetry (DSC) profile having a characteristic endotherm with an onset of about 92° C. and a peak of about 107° C., a characteristic endotherm with an onset of about 98° C. and a peak of about 119° C., and a characteristic endotherm with an onset of about 161° C. and a peak of about 173° C.

13. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form D), characterized by a powder X-ray diffraction pattern having characteristic peaks in degrees 2θ at about 6.3, 6.7, 8.4, 9.0, 10.0, 11.1, 11.3, 11.8, 13.4, 14.1, 14.4, and 14.8, wherein the powder X-ray diffraction pattern was obtained using Cu Kα radiation.

14. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form F), characterized by a powder X-ray diffraction pattern having characteristic peaks in degrees 2θ at about 5.3, 5.6, 6.1, 7.0, 8.1, 9.3, 10.6, 11.0, 12.5, 14.6, 17.3, and 20.1, wherein the powder X-ray diffraction pattern was obtained using Cu Kα radiation.

15. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form G), characterized by a powder X-ray diffraction pattern having characteristic peaks in degrees 2θ at about 4.2, 6.7, 7.6, 8.4, 8.9, 10.5, 12.6, 12.9, 13.3, 15.1, 21.4, and 21.7, wherein the powder X-ray diffraction pattern was obtained using Cu Kα radiation.

16. The crystalline form of claim 15, characterized by a differential scanning calorimetry (DSC) profile having a characteristic endotherm with an onset of about 166° C. and a peak of about 175° C., a characteristic endotherm with an onset of about 241° C. and a peak of about 245° C., a characteristic exotherm with an onset of about 249° C. and a peak of about 251° C., and a characteristic endotherm with an onset of about 267° C. and a peak of about 271° C.

17. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form H), characterized by a powder X-ray diffraction pattern having characteristic peaks in degrees 2θ at about 4.1, 6.2, 8.1, 9.3, 9.5, 10.2, 12.5, 14.7, 16.2, 16.8, 19.0, and 22.4, wherein the powder X-ray diffraction pattern was obtained using Cu Kα radiation.

18. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form I), characterized by a powder X-ray diffraction pattern having characteristic peaks in degrees 2θ at about 4.8, 6.1, 7.8, 8.1, 8.8, 9.1, 9.6, 10.3, 12.1, 13.2, 14.7, and 20.9, wherein the powder X-ray diffraction pattern was obtained using Cu Kα radiation.

19. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form J), characterized by a powder X-ray diffraction pattern having characteristic peaks in degrees 2θ at about 5.2, 6.3, 6.5, 7.8, 8.1, 8.3, 8.5, 10.6, 11.2, 14.2, 14.7, and 17.4, wherein the powder X-ray diffraction pattern was obtained using Cu Kα radiation.

20. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form K), characterized by a powder X-ray diffraction pattern having characteristic peaks in degrees 2θ at about 5.5, 7.1, 8.7, 9.3, 10.6, 10.8, 11.5, 13.3, 14.0, 14.9, 16.2, and 21.1, wherein the powder X-ray diffraction pattern was obtained using Cu Kα radiation.

21. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form L), characterized by a powder X-ray diffraction pattern having characteristic peaks in degrees 2θ at about 5.4, 7.0, 7.5, 8.9, 9.1, 9.6, 10.0, 11.1, 12.5, 15.0, and 18.2, wherein the powder X-ray diffraction pattern was obtained using Cu Kα radiation.

22. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form M), characterized by a powder X-ray diffraction pattern having characteristic peaks in degrees 2θ at about 4.8, 5.6, 7.3, 9.1, 9.6, 11.1, 12.4, 13.8, 14.5, 16.4, 16.7, and 19.4, wherein the powder X-ray diffraction pattern was obtained using Cu Kα radiation.

23. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form N), characterized by a powder X-ray diffraction pattern having characteristic peaks in degrees 2θ at about 6.5, 7.2, 8.9, 9.3, 9.8, 11.0, 12.0, 13.7, 14.4, 15.0, 20.2, and 20.9, wherein the powder X-ray diffraction pattern was obtained using Cu Kα radiation.

24. The crystalline form of claim 23, characterized by a differential scanning calorimetry (DSC) profile having a characteristic endotherm with an onset of about 39° C. and a peak of about 58° C., a characteristic endotherm with an onset of about 78° C. and a peak of about 94° C., a characteristic endotherm with an onset of about 171° C. and a peak of about 186° C., a characteristic endotherm with an onset of about 245° C. and a peak of about 249° C., and a characteristic endotherm with an onset of about 268° C. and a peak of about 271° C.

25. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form O), characterized by a powder X-ray diffraction pattern having characteristic peaks in degrees 2θ at about 5.6, 7.9, 8.8, 11.8, 14.2, 16.3, 16.8, 17.7, 19.8, 20.1, 21.3, and 23.1, wherein the powder X-ray diffraction pattern was obtained using Cu Kα radiation.

26. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form P), characterized by a powder X-ray diffraction pattern having characteristic peaks in degrees 2θ at about 5.6, 8.0, 8.9, 12.0, 14.4, 16.4, 16.9, 17.8, 19.9, 20.3, 21.5, and 23.3, wherein the powder X-ray diffraction pattern was obtained using Cu Kα radiation.

27. The crystalline form of claim 26, characterized by a differential scanning calorimetry (DSC) profile having a characteristic endotherm with an onset of about 47° C. and a peak of about 85° C., and a characteristic endotherm with an onset of about 179° C. and a peak of about 189° C.

28. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form Q), characterized by a powder X-ray diffraction pattern having characteristic peaks in degrees 2θ at about 7.5, 8.5, 9.8, 13.0, 14.8, 15.0, 15.8, 17.7, 18.6, 20.5, and 22.2, wherein the powder X-ray diffraction pattern was obtained using Cu Kα radiation.

29. The crystalline form of claim 28, characterized by a differential scanning calorimetry (DSC) profile having a characteristic endotherm with an onset of about 145° C. and a peak of about 152° C., a characteristic endotherm with an onset of about 244° C. and a peak of about 249, and a characteristic endotherm with an onset of about 266° C. and a peak of about 271° C.

30. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form R), characterized by a powder X-ray diffraction pattern having characteristic peaks in degrees 2θ at about 6.2, 8.0, 9.1, 9.6, 10.2, 10.9, 11.5, 12.3, 12.9, 14.0, 15.7, and 21.2, wherein the powder X-ray diffraction pattern was obtained using Cu Kα radiation.

31. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form S), characterized by a powder X-ray diffraction pattern having characteristic peaks in degrees 2θ at about 8.3, 9.4, 9.8, 10.7, 11.9, 13.5, 15.6, 20.1, 20.6, 21.8, 22.2, and 24.4, wherein the powder X-ray diffraction pattern was obtained using Cu Kα radiation.

32. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form T), characterized by a powder X-ray diffraction pattern having characteristic peaks in degrees 2θ at about 5.3, 6.5, 8.0, 8.5, 10.7, 11.3, 13.0, 13.5, 14.3, 14.7, 17.4, and 21.0, wherein the powder X-ray diffraction pattern was obtained using Cu Kα radiation.

33. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form U), characterized by a powder X-ray diffraction pattern having characteristic peaks in degrees 2θ at about 8.1, 9.5, 9.8, 12.7, 16.2, 16.9, 18.9, 19.5, 20.1, 21.2, 22.0, and 25.5, wherein the powder X-ray diffraction pattern was obtained using Cu Kα radiation.

34. The crystalline form of claim 33, characterized by a differential scanning calorimetry (DSC) profile having a characteristic endotherm with an onset of about 246° C. and a peak of about 250° C., a characteristic exotherm with an onset of about 253° C. and a peak of about 255° C., and a characteristic endotherm with an onset of about 270° C. and a peak of about 274° C.

35. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form V), characterized by a powder X-ray diffraction pattern having characteristic peaks in degrees 2θ at about 8.2, 9.0, 10.1, 13.5, 13.9, 14.9, 18.7, 19.6, 20.4, 21.4, 21.7, and 24.1, wherein the powder X-ray diffraction pattern was obtained using Cu Kα radiation.

36. The crystalline form of claim 35, characterized by a differential scanning calorimetry (DSC) profile having a characteristic endotherm with an onset of about 165° C. and a peak of about 176° C., a characteristic exotherm with an onset of about 182° C. and a peak of about 191° C., a characteristic endotherm with an onset of about 198° C. and a peak of about 204° C., a characteristic endotherm with an onset of about 247° C. and a peak of about 251° C., a characteristic exotherm with an onset of about 255° C. and a peak of about 257° C., and a characteristic endotherm with an onset of about 270° C. and a peak of about 274° C.

37. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form W), characterized by a powder X-ray diffraction pattern having characteristic peaks in degrees 2θ at about 6.4, 7.1, 8.9, 9.4, 9.8, 10.6, 10.8, 11.8, 13.5, 14.2, 15.1, and 16.6, wherein the powder X-ray diffraction pattern was obtained using Cu Kα radiation.

38. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form X), characterized by a powder X-ray diffraction pattern having characteristic peaks in degrees 2θ at about 4.5, 7.1, 9.1, 10.5, 11.8, 13.7, 14.0, and 16.1, wherein the powder X-ray diffraction pattern was obtained using Cu Kα radiation.

39. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form Y), characterized by a powder X-ray diffraction pattern having characteristic peaks in degrees 2θ at about 6.9, 8.9, 10.3, 14.4, 20.5, and 20.6, wherein the powder X-ray diffraction pattern was obtained using Cu Kα radiation.

40. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form Z), characterized by a powder X-ray diffraction pattern having characteristic peaks in degrees 2θ at about 5.5, 8.4, 8.9, 9.3. 9.7, 11.7, 11.9, 14.7, 14.8, 17.9, 20.1, and 22.0, wherein the powder X-ray diffraction pattern was obtained using Cu Kα radiation.

41. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form α), characterized by a powder X-ray diffraction pattern having characteristic peaks in degrees 2θ at about 4.3, 8.5, 9.2, 9.3, 9.6, 9.7, 12.4, 12.8, 14.7, 15.4, 16.0, and 17.0, wherein the powder X-ray diffraction pattern was obtained using Cu Kα radiation.

42. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form β), characterized by a powder X-ray diffraction pattern having characteristic peaks in degrees 2θ at about 5.8, 6.7, 8.9, 10.1, 12.1, 13.4, 14.7, 16.8, 17.8, 20.5, 21.0, and 24.3, wherein the powder X-ray diffraction pattern was obtained using Cu Kα radiation.

43. A crystalline form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine (Form γ), characterized by a powder X-ray diffraction pattern having characteristic peaks in degrees 2θ at about 6.3, 6.6, 7.8, 8.4, 8.9, 10.4, 10.6, 12.6, 13.3, 20.3, 21.1, and 21.7, wherein the powder X-ray diffraction pattern was obtained using Cu Kα radiation.

44. A substantially amorphous form of (S)-12-fluoro-4-(1-methyl-1H-pyrazol-5-yl)-7a,8,13,14-tetrahydro-7H-[1,2,4]triazolo[4′,3′:1,6]pyrido[3,2-b]benzofuro[4,3-fg][1,4]oxazonine.

45. A pharmaceutical composition comprising a crystalline form of any one of claims 1-43 or the amorphous form of claim 44, and a pharmaceutically acceptable excipient.

46. A pharmaceutical composition formed from the crystalline form of any one of claims 1-43.

47. A pharmaceutical composition comprising the crystalline Form E of any one of claims 1-6, and a pharmaceutically acceptable excipient.

48. A pharmaceutical composition formed from the crystalline Form E of any one of claims 1-6.

49. The pharmaceutical composition of any one of claims 45-48, wherein the composition is a formulation for oral administration.

50. A drug substance comprising at least a detectable amount of the crystalline form of any one of claims 1-43.

51. A drug substance comprising a substantially pure crystalline form of any one of claims 1-43.

52. A drug substance comprising a substantially pure crystalline Form E of any one of claims 1-6.
